SEQUENCE LISTING (19 sequences)

SEQ ID NO: 1 - DNA, 4610 bp, Homo sapiens
gcggcagcggcggcggctgaggagggcccggcctgcgagagcctcagtgggagccggctcagccctcggccaccatgtcggcgccgtcggaggaggaggagtacgcgcggctggtgatggaggcgcagccggagtggctgcgcgccgaggtgaagcggctgtcccacgagctggccgagaccacgcgtgagaagatccaggcggccgagtacgggctggcggtgctcgaggagaagcaccagctcaagctgcagttcgaggagctcgaggtggactatgaggctatccgcagcgagatggagcagctcaaggaggcctttggacaagcacacacaaaccacaagaaggtggctgctgacggagagagccgggaggagagcctgatccaggagtcggcctccaaggagcagtactacgtgcggaaggtgctagagctgcagacggagctgaagcagttgcgcaatgtcctcaccaacacgcagtcggagaatgagcgcctggcctctgtggcccaggagctgaaggagatcaaccagaatgtggagatccagcgtggccgcctgcgggatgacatcaaggagtacaaattccgggaagctcgtctgctgcaggactactcggaactggaggaggagaacatcagcctgcagaagcaagtgtctgtgctcagacagaaccaggtggagtttgagggcctcaagcatgagatcaagcgtctggaggaggagaccgagtacctcaacagccagctggaggatgccatccgcctcaaggagatctcagagcggcagctggaggaggcgctggagaccctgaagacggagcgcgaacagaagaacagcctgcgcaaggagctgtcacactacatgagcatcaatgactccttctacaccagccacctgcatgtctcgctggatggcctcaagttcagtgacgatgctgccgagcccaacaacgatgccgaggccctggtcaatggctttgagcacggcggcctggccaagctgccactggacaacaagacctccacgcccaagaaggagggcctcgcaccgccctcccccagcctcgtctccgacctactcagtgagctcaacatctctgagatccagaagctgaagcagcagctgatgcagatggagcgggaaaaggcgggcctgctggcaacgctgcaggacacacagaagcagctggagcacacgcggggctccctgtcagaacagcaggagaaggtgacccgcctcacagagaatctgagtgccctgcggcgcctgcaggccagcaaggagcggcagacagccctggacaacgagaaggaccgtgacagccatgaggatggggactactacgaggtggacatcaacgggcctgagatcttggcctgcaagtaccatgtggctgtggctgaggctggcgagctccgcgagcagctcaaggcactgcgcagcacgcacgaggctcgtgaggcccagcacgccgaggagaagggccgctatgaggctgagggccaggcactcacggagaaggtctccctgctagagaaggccagccgccaggaccgcgagctgctggcccggctggagaaggagctaaagaaggtgagcgacgtcgccggcgagacacagggcagcctgagtgtggcccaggatgagctggtgaccttcagtgaggagctggccaatctctaccaccacgtgtgcatgtgcaacaatgagacacccaaccgtgtcatgctggactactaccgcgagggccagggcggggccggccgcaccagtcccgggggccgcaccagccccgaggcgcgtggccggcgctcacccatcctcctacccaaggggctgctggctcctgaggcgggccgagcagatggtgggacgggggacagcagcccctcgcctggctcctcactgccatcacccctgagtgacccacgccgggagcccatgaacatctacaacctgatcgctatcatccgtgaccagatcaagcacctgcaggcagccgtggaccgcaccacggagctgtcacgccagcgcattgcctctcaggagctgggccccgccgtggacaaggacaaggaagcgcttatggaggagatcctcaagctgaagtcgctgctcagcaccaagcgggagcagatcaccacgctgcgcactgtgctcaaggccaacaagcagacggccgaggtggcccttgccaacctgaagagcaagtatgagaatgagaaggccatggttaccgagaccatgatgaagctgcgcaatgagctcaaggccctcaaggaggacgcagccaccttctcctcgctgcgtgctatgtttgccaccaggtgtgacgagtacattacacagctggatgagatgcagcggcagctggcggctgctgaggacgagaagaagacgctgaactcgctgctgcgcatggccatccagcagaagctggcgctgacccagcggctggagctgctcgagctggaccatgagcagacccggcgtggccgtgccaaagccgccccgaagaccaagccagccacaccgagcctgtagagtagctgccaggaggacttggccacccggccctgtcacactgcagccccttccccttccctctcgtggcccacaaggaggaaggaagggcaacctaaaagcccacttagaaactttttggatatgccactgcaattcttttcaaaatagcattccccaggtttttaatgggaggaaaaaaagctttaatgttgagcatgctgcgagctgctgcgtggaaaggcctctgtatgggccgaagacccttcttccctggctgccaggctcgccaggagcccactggaaacgcccaccacgggggctccttgttacacatgttctttttttatccgatcaacctgtgcacttttgatattttgatattatatttgcttccttaattcctcgcgtagagacggtctcaggtgccgtggtctatgctcgtggtcctgtagctgtccgcctcagctcccaccgtgtttgtctggtgtcagcacgaggcagagctgtgtgctccatagcgtgtagctttagactcggagatgagtgctttgacccagcgaggagctcagctaagtgtatccacgctgtggttcagcagcctttagatcatacggcattgtggttcatgtttgaaattacagattttaaatgccatgttcattaagaaatccagggtattcagattctggggtttttcatattgtattattattattcttaggaatagttcaatgtaacaagaagaaaacttgacctttgctctggttaaaacagtaataggcacttgaaaaaaaaagataaattattgaatgagtagtattacctacaaattccagaattttctgggttttaggacgttgtgaagcatgactgattaacagaattttatacaactgtaccaataaaattccaaattggaattgttttgttactctggttgttgtgccaaattgtggtacacttagaaaattctacagtcgtcgatttttagggtgttctctttcaacacctttttgttagtaatcattgccagtagtgccttcatcagttaagggaggtgtcccagcacagatcattctcaaaagcgagcagggaagagctagtgggcatgctgaaggccagcgtggacagcaggtgaggcaggtgctcctcacacccagacctgggcatcttcattgagggaaagaaaacagtcattgtgcaaaattctgttagtcagtgattctttacttgcaaattcaggggcttagaaaatgaaagcaaacacaaaaccttgagtgtgctttgggaaccaaatggaccttctgggacaagctgagcaagctgtatgaacgccacgtttgtgaagagctgagggtatcaggagggccgacgctgtgttggcatgcgcagtaggggatgagggttagccatagtattctttgcaaatgtgaaagcgagacattatatcttctcttgcttggtgtaactaatcactgttaatttcaggaaacagaactcattaaaactccttagcaaaccaggtctacatcctgttttgtttgctgagtgaggttagtgggagtggtcaaattggtactcttggaggaagaaaaactgtccttccttctccaaaaaaggaaaaattataataatataaatgacaaaaataaaagaattctgtttcctggaataagcatttcttattcctagttgtagggactcctatttttaccttccgttacagtgttgattcataagaaatattgttacatttgagataacttcatctgtatggggtatttatttgcaatgatgtctgagtactgtattttttctgtgcattaccttagtgtcagaatgttggtctttattttaaagtcatatgcatgttctcctgccaaggaacctttacacagacccaaacaaaaaaataataatcaaatgccttcaatttctgagaaaatgaggcagagcatggaaaaggaataggaaggagaaattaattgagattttcaggacacagacatatgatgtgaatgcctacaaagccagtgtgcataggaacagtgggcctgggtaaagagtcacattggtagg

SEQ ID NO: 2 - protein, 824 aa, Homo sapiens
MSAPSEEEEYARLVMEAQPEWLRAEVKRLSHELAETTREKIQAAEYGLAVLEEKHQLKLQFEELEVDYEAIRSEMEQLKEAFGQAHTNHKKVAADGESREESLIQESASKEQYYVRKVLELQTELKQLRNVLTNTQSENERLASVAQELKEINQNVEIQRGRLRDDIKEYKFREARLLQDYSELEEENISLQKQVSVLRQNQVEFEGLKHEIKRLEEETEYLNSQLEDAIRLKEISERQLEEALETLKTEREQKNSLRKELSHYMSINDSFYTSHLHVSLDGLKFSDDAAEPNNDAEALVNGFEHGGLAKLPLDNKTSTPKKEGLAPPSPSLVSDLLSELNISEIQKLKQQLMQMEREKAGLLATLQDTQKQLEHTRGSLSEQQEKVTRLTENLSALRRLQASKERQTALDNEKDRDSHEDGDYYEVDINGPEILACKYHVAVAEAGELREQLKALRSTHEAREAQHAEEKGRYEAEGQALTEKVSLLEKASRQDRELLARLEKELKKVSDVAGETQGSLSVAQDELVTFSEELANLYHHVCMCNNETPNRVMLDYYREGQGGAGRTSPGGRTSPEARGRRSPILLPKGLLAPEAGRADGGTGDSSPSPGSSLPSPLSDPRREPMNIYNLIAIIRDQIKHLQAAVDRTTELSRQRIASQELGPAVDKDKEALMEEILKLKSLLSTKREQITTLRTVLKANKQTAEVALANLKSKYENEKAMVTETMMKLRNELKALKEDAATFSSLRAMFATRCDEYITQLDEMQRQLAAAEDEKKTLNSLLRMAIQQKLALTQRLELLELDHEQTRRGRAKAAPKTKPATPSL

SEQ ID NO: 3 - DNA, 2940 bp, Homo sapiens
atgtcggtgctgggcgagtacgagcgacactgcgattccatcaactcggactttgggagcgagtccgggggttgcggggactcgagtccggggcctagcgccagtcaggggccgcgagccggcggcggcgcggcggagcaggaggaactgcactacatccccatccgcgtcctgggccgcggcgccttcggggaagccacgctgtaccgccgcaccgaggatgactcactggttgtgtggaaggaagtcgatttgacccggctgtctgagaaggaacgtcgtgatgccttgaatgagattgttattctggcactgctgcagcatgacaacattattgcctactacaatcacttcatggacaataccacgctgctgattgagctggaatattgtaatggagggaacctgtatgacaaaatccttcgtcagaaggacaagttgtttgaggaagagatggtggtgtggtacctatttcagattgtttcagcagtgagctgcatccataaagctggaatccttcatagagatataaagacattaaatatttttctgaccaaggcaaacctgataaaacttggagattatggcctagcaaagaaacttaattctgagtattccatggctgagacgcttgtgggaaccccatattacatgtctccagagctctgtcaaggagtaaagtacaatttcaagtctgatatctgggcagttggctgcgtcatttttgaactgcttaccttaaagaggacgtttgatgctacaaacccacttaacctgtgtgtgaagatcgtgcaaggaattcgggccatggaagttgactctagccagtactctttggaattgatccaaatggttcattcgtgccttgaccaggatcctgagcagagacctactgcagatgaacttctagatcgccctcttctcaggaaacgcaggagagagatggaggaaaaagtcactctgcttaatgcacctacaaagagaccaaggtcaagcactgtgactgaagcacccattgctgtagtaacatcacgaaccagtgaagtctatgtttggggtggtggaaaatccaccccccagaaactggatgttatcaagagtggctgtagtgcccggcaggtctgtgcagggaatacccactttgctgtggtcacagtggagaaggaactgtacacttgggtgaacatgcaaggaggcactaaactccatggtcagctgggccatggagacaaagcctcctatcgacagccaaagcatgtggaaaagttgcaaggcaaagctatccatcaggtgtcatgtggtgatgatttcactgtctgtgtgactgatgagggtcagctctatgccttcggatcagattattatggctgcatgggggtggacaaagttgctggccctgaagtgctagaacccatgcagctgaacttcttcctcagcaatccagtggagcaggtctcctgtggagataatcatgtggtggttctgacacgaaacaaggaagtctattcttggggctgtggcgaatatggacgactgggtttggattcagaagaggattattatacaccacaaaaggtggatgttcccaaggccttgattattgttgcagttcaatgtggctgtgatgggacatttctgttgacccagtcaggcaaagtgctggcctgtggactcaatgaattcaataagctgggtctgaatcagtgcatgtcgggaattatcaaccatgaagcataccatgaagttccctacacaacgtcctttaccttggccaaacagttgtccttttataagatccgtaccattgccccaggcaagactcacacagctgctattgatgagcgaggccggctgctgacctttggctgcaacaagtgtgggcagctgggcgttgggaactacaagaagcgtctgggaatcaacctgttggggggaccccttggtgggaagcaagtgatcagggtctcctgcggtgatgagtttaccattgctgccactgatgataatcacatttttgcctggggcaatggtggtaatggccgcctggcaatgacccccacagagagaccacatggctctgatatctgtacctcatggcctcggcctatttttggatctctgcatcatgtcccggacctgtcttgccgtggatggcataccattctcatcgttgagaaagtattgaattctaagaccatccgttccaatagcagtggcttatccattggaactgtgtttcagagctctagcccgggaggaggcggcgggggcggcggtggtgaagaagaggacagtcagcaggaatctgaaactcctgacccaagtggaggcttccgaggaacaatggaagcagaccgaggaatggaaggtttaatcagtcccacagaggccatggggaacagtaatggggccagcagctcctgtcctggctggcttcgaaaggagctggaaaatgcagaatttatccccatgcctgacagcccatctcctctcagtgcagcgttttcagaatctgagaaagataccctgccctatgaagagctgcaaggactcaaagtggcctctgaagctcctttggaacacaaaccccaagtagaagcctcgtcacctcggctgaatcctgcagtaacctgtgctgggaagggaacaccactgactcctcctgcgtgtgcgtgcagctctctgcaggtggaggttgagagattgcagggtctggtgttaaagtgtctggctgaacaacagaagctacagcaagaaaacctccagatttttacccaactgcagaagttgaacaagaaattagaaggagggcagcaggtggggatgcattccaaaggaactcagacagcaaaggaagagatggaaatggatccaaagcctgacttagattcagattcctggtgcctcctgggaacagactcctgtagacccagcctctag

SEQ ID NO: 4 - protein, 979 aa, Homo sapiens
MSVLGEYERHCDSINSDFGSESGGCGDSSPGPSASQGPRAGGGAAEQEELHYIPIRVLGRGAFGEATLYRRTEDDSLVVWKEVDLTRLSEKERRDALNEIVILALLQHDNIIAYYNHFMDNTTLLIELEYCNGGNLYDKILRQKDKLFEEEMVVWYLFQIVSAVSCIHKAGILHRDIKTLNIFLTKANLIKLGDYGLAKKLNSEYSMAETLVGTPYYMSPELCQGVKYNFKSDIWAVGCVIFELLTLKRTFDATNPLNLCVKIVQGIRAMEVDSSQYSLELIQMVHSCLDQDPEQRPTADELLDRPLLRKRRREMEEKVTLLNAPTKRPRSSTVTEAPIAVVTSRTSEVYVWGGGKSTPQKLDVIKSGCSARQVCAGNTHFAVVTVEKELYTWVNMQGGTKLHGQLGHGDKASYRQPKHVEKLQGKAIHQVSCGDDFTVCVTDEGQLYAFGSDYYGCMGVDKVAGPEVLEPMQLNFFLSNPVEQVSCGDNHVVVLTRNKEVYSWGCGEYGRLGLDSEEDYYTPQKVDVPKALIIVAVQCGCDGTFLLTQSGKVLACGLNEFNKLGLNQCMSGIINHEAYHEVPYTTSFTLAKQLSFYKIRTIAPGKTHTAAIDERGRLLTFGCNKCGQLGVGNYKKRLGINLLGGPLGGKQVIRVSCGDEFTIAATDDNHIFAWGNGGNGRLAMTPTERPHGSDICTSWPRPIFGSLHHVPDLSCRGWHTILIVEKVLNSKTIRSNSSGLSIGTVFQSSSPGGGGGGGGGEEEDSQQESETPDPSGGFRGTMEADRGMEGLISPTEAMGNSNGASSSCPGWLRKELENAEFIPMPDSPSPLSAAFSESEKDTLPYEELQGLKVASEAPLEHKPQVEASSPRLNPAVTCAGKGTPLTPPACACSSLQVEVERLQGLVLKCLAEQQKLQQENLQIFTQLQKLNKKLEGGQQVGMHSKGTQTAKEEMEMDPKPDLDSDSWCLLGTDSCRPSL

SEQ ID NO: 5 - DNA, 21 bp, Mus sp.
ccggtggatgtggaatgtgtg

SEQ ID NO: 6 - DNA, 22 bp, Mus sp.
caaagccaaggttcattcggtg

SEQ ID NO: 7 - DNA, 22 bp, Mus sp.
gccctgaatgaactgcaggacg

SEQ ID NO: 8 - DNA, 22 bp, Mus sp.
cacgggtagccaacgctatgtc

SEQ ID NO: 9 - DNA, 21 bp, Mus sp.
cttccgcttccacgacactcg

SEQ ID NO: 10 - DNA, 21 bp, Mus sp.
ctcaatggcctcagacgccag

SEQ ID NO: 11 - DNA, 20 bp, Homo sapiens
gcctttggacaagcacacac

SEQ ID NO: 12 - DNA, 19 bp, Homo sapiens
ctccttcagctcctgggcc

SEQ ID NO: 13 - DNA, 30 bp, Homo sapiens
cgagctgctctatagactgctgggtagtcc

SEQ ID NO: 14 - DNA, 30 bp, Homo sapiens
taacagaggtggcttatgagtatttcttcc

SEQ ID NO: 15 - DNA, 20 bp, Homo sapiens
aaaccacaagaaggtggctg

SEQ ID NO: 16 - DNA, 22 bp, Homo sapiens
aggtgaagcggctgtcccacga

SEQ ID NO: 17 - DNA, 22 bp, Homo sapiens
ctccttcagctcctgggccaca

SEQ ID NO: 18 - protein, 821 aa, Homo sapiens
MAAEEVLQTVDHYKTEIERLTKELTETTHEKIQAAEYGLVVLEEKLTLKQQYDELEAEYDSLKQELEQLKEAFGQSFSIHRKVAEDGETREETLLQESASKEAYYLGKILEMQNELKQSRAVVTNVQAENERLTAVVQDLKENNEMVELQRIRMKDEIREYKFREARLLQDYTELEEENITLQKLVSTLKQNQVEYEGLKHEIKRFEEETVLLNSQLEDAIRLKEIAEHQLEEALETLKNEREQKNNLRKELSQYISLNDNHISISVDGLKFAEDGSEPNNDDKMNGHIHGPLVKLNGDYRTPTLRKGESLNPVSDLFSELNISEIQKLKQQLMQVEREKAILLANLQESQTQLEHTKGALTEQHERVHRLTEHVNAMRGLQSSKELKAELDGEKGRDSGEEAHDYEVDINGLEILECKYRVAVTEVIDLKAEIKALKEKYNKSVENYTDEKAKYESKIQMYDEQVTSLEKTTKESGEKMAHMEKELQKMTSIANENHSTLNTAQDELVTFSEELAQLYHHVCLCNNETPNRVMLDYYRQSRVTRSGSLKGPDDPRGLLSPRLARRGVSSPVETRTSSEPVAKESTEPSKEPSPTKTPTISPVITAPPSSPVLDTSDIRKEPMNIYNLNAIIRDQIKHLQKAVDRSLQLSRQRAAARELAPMIDKDKEALMEEILKLKSLLSTKREQIATLRAVLKANKQTAEVALANLKNKYENEKAMVTETMTKLRNELKALKEDAATFSSLRTMFATRCDEYVTQLDEMQRQLAAAEDEKKTLNTLLRMAIQQKLALTQRLEDLEFDHEQSRRSKGKLGKSKIGSPKV

SEQ ID NO: 19 - protein, 868 aa, Homo sapiens
NTHLEAQLQKAEEAGAELQADLRDIQEEKEEIQKKLSESRHQQEAATTQLEQLHQEAKRQEEVLARAVQEKEALVREKAALEVRLQAVERDRQDLAEQLQGLSSAKELLESSLFEAQQQNSVIEVTKGQLEVQIQTVTQAKEVIQGEVRCLKLELDTERSQAEQERDAAARQLAQAEQEGKTALEQQKAAHEKEVNQLREKWEKERSWHQQELAKALESLEREKMELEMRLKEQQTEMEAIQAQREEERTQAESALCQMQLETEKERVSLLETLLQTQKELADASQQLERLRQDMKVQKLKEQETTGILQTQLQEAQRELKEAARQHRDDLAALQEESSSLLQDKMDLQKQVEDLKSQLVAQDDSQRLVEQEVQEKLRETQEYNRIQKELEREKASLTLSLMEKEQRLLVLQEADSIRQQELSALRQDMQEAQGEQKELSAQMELLRQEVKEKEADFLAQEAQLLEELEASHITEQQLRASLWAQEAKAAQLQLRLRSTESQLEALAAEQQPGNQAQAQAQLASLYSALQQALGSVCESRPELSGGGDSAPSVWGLEPDQNGARSLFKRGPLLTALSAEAVASALHKLHQDLWKTQQTRDVLRDQVQKLEERLTDTEAEKSQVHTELQDLQRQLSQNQEEKSKWEGKQNSLESELMELHETMASLQSRLRRAELQRMEAQGERELLQAAKENLTAQVEHLQAAVVEARAQASAAGILEEDLRTARSALKLKNEEVESERERAQALQEQGELKVAQGKALQENLALLTQTLAEREEEVETLRGQIQELEKQREMQKAALELLSLDLKKRNQEVDLQQEQIQELEKCRSVLEHLPMAVQEREQKLTVQREQIRELEKDRETQRNVLEHQL

(19) United States
(12) Patent Application Publication
(10) Pub. No.: US 2002/0182656 A1
(43) Pub. Date: Dec. 5, 2002

(54) Methods for regulating vascularization using GEF containing NEK-like kinase (GNK)

(76) Inventors: Timothy A. Bird, Bainbridge Island, WA (US); Jacques J. Peschon, Seattle, WA (US); John E. Sims, Seattle, WA (US); G. Duke Virca, Bellevue, WA (US); Cynthia R. Willis, Clinton, WA (US)

Correspondence Address: IMMUNEX CORPORATION, LAW DEPARTMENT, 51 UNIVERSITY STREET, SEATTLE, WA, 98101

(21) Appl. No.: 09/884,001

(22) Filed: Jun. 18, 2001

Related U.S. Application Data
(63) Continuation of application No. PCT/US99/29989, filed on Dec. 17, 1999.
(60) Provisional application No. 60/113,003, filed on Dec. 18, 1998.

(51) Int. Cl. G01N 33/574; C12N 9/12; C07H 21/04; C12P 21/02; C12N 5/06

(52) U.S. Cl. 435/7.23; 435/194; 435/320.1; 435/325; 435/69.1; 536/23.2

(57) ABSTRACT

The present invention provides a novel use for GNK in treating pathological conditions related to angiogenesis. The present invention also provides isolated DNA encoding sGNK, expression vectors comprising the isolated DNA, and a method for producing sGNK by cultivating host cells containing the expression vectors under conditions appropriate for expression of the sGNK. Antibodies directed against sGNK or an immunogenic fragment thereof are also disclosed. The sGNK, which is a physiological substrate of GNK and co-purifies with GNK on gel filtration chromatography, may also be useful in treating vascularization abnormalities.

### CROSS-REFERENCE TO RELATED APPLICATION

The present application claims the benefit of U.S. Provisional Application No. 60/113,003, filed Dec. 18, 1998, which is hereby incorporated by reference.

### FIELD OF THE INVENTION

The invention is generally directed toward the use of a novel protein kinase, GEF containing NEK-like Kinase (GNK), previously designated IL-1/TNF-α activated kinase (ITAK), and its physiological substrate, sGNK, in regulating vascularization. More specifically, the invention is directed to stimulating blood vessel development using the GNK and its agonists, and to inhibiting inappropriate blood vessel development using antagonists of GNK.

### BACKGROUND OF THE INVENTION

The protein kinases regulate many different cell cycle, differentiation and signaling processes by catalyzing the addition of phosphate groups to protein substrates. Reversible protein phosphorylation is the main mechanism for regulating eukaryotic cell activities. Proteins are generally activated by the kinase-catalyzed transfer of high energy phosphate from adenosine triphosphate (ATP) or guanine triphosphate (GTP), referred to as phosphorylation, and deactivated by the reverse process, referred to as dephosphorylation, the removal of phosphate group from activated proteins by enzymes known as protein phosphatases. While some kinases act on a single substrate to bring about their biological effect, others are involved in complex biological networks or signaling pathways in which kinase-catalyzed phosphorylation triggers a cascade effect with multiple “downstream” events, which may include the activation of additional kinases.

There are three primary types of kinases, categorized by the amino acid residue to which they catalytically transfer a phosphate group. Serine/threonine kinases transfer phosphate molecules to the alcoholic moiety of either serine or threonine residues within a polypeptide. Tyrosine kinases catalyze the transfer of phosphate to the phenolic moiety of tyrosine residues. Dual specificity kinases are capable of catalyzing the transfer of phosphate to serine, threonine, or tyrosine residues within a polypeptide.

Kinases can respond to extracellular signals, such as hormones, growth factors, pheromones, cytokines, or neurotransmitters. These extracellular signaling molecules, which allow cell-to-cell signaling, bind to specific receptors on the cell membrane, in the cytosol or in the nucleus (e.g., lipophilic hormones), forming receptor-ligand complexes. Kinase activity is also induced in response to environmental conditions such as ultraviolet radiation or stress, or in response to cell-cycle stimuli.

When induced, kinases activate a variety of substrate molecules including enzymes, regulatory proteins, receptors, cytoskeletal proteins, transcription factors, ion channels and pumps. There are also kinases which are capable of phosphorylating themselves, a process known as autophosphorylation. In all forms of phosphorylation, the biological activity of each substrate is altered as the result of phosphorylation. Phosphorylated substrate molecules generally remain active until they are “turned off” by phosphatases which dephosphorylate them.

Protein kinases play a significant role in both B-cell and T-cell activation, as well as many phases of the immune response. The biological activity of many cytokines, including interleukin 1 (IL-1) and tumor necrosis factor (TNF), depend heavily on kinase-catalyzed protein phosphorylation. The binding of either of these cytokines to their respective receptors is known to induce rapid phosphorylation of several cytosolic proteins, such as the inhibitor of nuclear factor kappa B (NF-κB), heat shock protein 27 (hsp27), and mitogen-activated protein kinases (MAPK). (Geusdon et al., J. Biol. Chem. 272:30017, 1997).

Protein kinases have also been shown to be significantly involved in cell cycle regulation. The centrosome, which plays a key role in cell division, undergoes a series of morphological and functional changes during the cell cycle. Centrosomes, which gives rise to the poles of the mitotic spindle apparatus, consist of a pair of centrioles surrounded by an amorphous structure known as pericentriolar material (PCM) from which microtubules are nucleated. In late G₁/S phase of the cell cycle, centrioles undergo semi-conservative replication. During the S and G₂ phases, the centrosome enlarges and “matures” as additional PCM proteins are recruited. The duplicated centrosomes physically separate and migrate to opposite ends of the nucleus at the transition of the G₂ to M phases. Additionally, the centrosomes abruptly increase their microtubule-nucleating capacity at the onset of mitosis. Many of these events are believe to be the result of kinase-catalyzed phosphorylation of critical centrosomal proteins, as protein kinases have been implicated in the duplication, maturation and separation of centrosomes during the cell cycle and are thought to regulate the centrosomal microtubule nucleation capacity. (Fry et al., J. Cell Biol., 141:1563, 1998).

As our understanding of kinases and signal transduction pathways increases, means for interceding in the progression of certain disease states are beginning to emerge. Once a kinase and its substrate are identified, it will be possible to either inhibit activity through the use of an antagonist or to enhance the kinase's activity using an agonist. Kinase activity may also be enhanced by increasing expression of the kinase gene or by the addition of exogenous kinase. Through activating or inhibiting kinase activity, biological effects can be regulated such that many pathologic conditions may be improved or remedied.

One area in which the modulation of kinase activity may play a role is in the vascular system. The regulated development and maintenance of a functional vascular system is essential for fetal and post-natal life. For example, mouse mutations that block or compromise vasculogenesis (i.e., the development of vessels from vascular progenitor cells) or angiogenesis (i.e., the formation of capillaries from pre-existing vessels) generally result in embryonic lethality at various stages of development between e8.5 and e13.5 (days of embryonic development). During adult life, the regulation of vascularization is critical in normal organ homeostasis and during wound repair. In contrast, excessive vascularization is associated with, and contributes to, a number of inflammatory disorders, including arthritis, psoriasis and diabetic retinopathy. Additionally, the survival of tumors beyond a finite size is strictly dependent upon recruitment of blood vessels into a tumor site. Thus agents that promote or attenuate blood vessel development have multiple applications in the treatment of vascular disorders or diseases in which dysregulated vascularization plays a critical role in pathogenesis.

Consequently, there is a continuing need for substances and methods for regulating vascularization processes. The discovery that the novel kinase, GNK, and by implication its physiological substrate sGNK, affects vascular development now provides us with a means for regulating vascularization. The present invention provides methods for regulating vascularization, including isolated novel kinase and sGNK as well as agonists and antagonists of their biological activity.

### SUMMARY OF THE INVENTION

The present invention provides a novel use for GNK in regulating vascularization, for example, in treating pathological conditions related to angiogenesis or in circumstances where it is important to induce vasculogenesis. Methods for treating vascularization disorders, both undervascularization and inappropriate blood vessel development, using GNK, its agonists and antagonists are disclosed. The present invention also provides isolated DNA encoding sGNK, expression vectors comprising the isolated DNA, and a method for producing sGNK by cultivating host cells containing the expression vectors under conditions appropriate for expressing sGNK. Antibodies directed against sGNK, or an immunogenic fragment thereof, are also disclosed. The sGNK, which is a physiological substrate of GNK and co-purifies with GNK through an ammonium sulphate precipitation and seven subsequent chromatographic purification steps, may also be useful in treating vascularization abnormalities.

The invention includes an isolated human nucleic acid molecule comprising the DNA sequence of SEQ ID NO: 1 and isolated polypeptides having the amino acid sequence of SEQ ID NO: 2 and variants thereof due to the addition, deletion, or substitution of one or more amino acids. The invention also encompasses nucleic acid molecules that hybridize with the DNA sequence of SEQ ID NO: 1. A preferred set of hybridization conditions are those of moderate stringency, i.e., in 50% formamide and 6× SSC, at 42° C. with washing conditions of 0.5× SSC, 0.1% sodium dodecyl sulfate (SDS) at 60° C.

The present invention also encompasses an isolated human nucleic acid sequence encoding a sufficient number of amino acids of SEQ ID NO: 4 to confer on a GNK polypeptide the potential to regulate vascularization in mammals and an isolated human nucleic acid molecule comprising a sufficient number of nucleotides from SEQ ID NO: 3 to encode a GNK polypeptide that enhances vascularization.

Expression vectors comprising the sGNK DNA sequences are provided, as well as methods for producing recombinant sGNK by culturing host cells under conditions appropriate for expressing sGNK, and for expressing a polypeptide having vascularization regulatory activity are provided.

The present invention also provides methods for identifying a compound that modulates GNK-sGNK interaction or phosphorylation of sGNK by GNK. These methods comprise contacting candidate compounds with GNK and sGNK under conditions that allow the interaction or the phosphorylation to occur and then measuring the ability of the candidate compound to modulate interaction between GNK and sGNK or phosphorylation of sGNK by GNK. Compounds identified by these methods will be useful for further study and may have many in vivo and in vitro applications.

Also provided is a method of identifying a compound that modulates vascularization comprising contacting a candidate compound with GNK or sGNK and measuring the ability of the compound to modulate a biological activity of GNK or sGNK. Compounds identified by this method will be useful for further study and may have many in vivo and in vitro applications.

The present invention also encompasses nonhuman transgenic embryos, fetuses, and animals that are heterozygous for a GNK targeted mutation, nonhuman GNK-deficient embryos and fetuses produced by crossing such heterozygous animals, and cells from these embryos, fetuses, and animals. Cells deficient in GNK or sGNK are also provided.

Additional features and advantages of the invention will be set forth in the description which follows, and in part will be apparent from the description, or may be learned by practice of the invention. The objectives and other advantages of the invention will be realized and attained by the methods, the recombinant vectors and proteins, and the pharmaceutical compositions particularly pointed out in the written description and claims hereof, as well as the appended drawings.

It is to be understood that both the foregoing general description and the following detailed description are exemplary and explanatory and are intended to provide further explanation of the invention as claimed. The accompanying figures are included to provide a further understanding of the invention and are incorporated in and constitute a part of this specification. These figures illustrate several embodiments of the invention and, together with the description, serve to explain the principles of the invention.

Throughout this specification many documents are cited. All of these documents are hereby specifically incorporated by reference.

### BRIEF DESCRIPTION OF THE DRAWINGS

This invention will be more fully described with reference to the drawings in which:

FIG. 1 is the nucleotide sequence of sGNK, SEQ ID NO: 1, including the coding sequence (base pair 75-2549), the 5′ untranslated region (UTR) and the 3′ UTR;

FIG. 2 is the predicted amino acid sequence of sGNK, SEQ ID NO: 2;

FIG. 3 is the nucleotide sequence of GNK, SEQ ID NO: 3;

FIG. 4 is the predicted amino acid sequence of GNK, SEQ ID NO: 4;

FIG. 5 is the schematic representation of the domains of GNK and sGNK that depicts for GNK the serine/threonine kinase domain, the GEF homology domain, the gly/glu-rich linker, and the unique region, and for sGNK, predicted regions of moderate or high coiled-coil probability;

FIG. 6 is a tabular representation of the domains and structural features of GNK, SEQ ID NO: 4, that depicts the putative kinase domain (residues 44-315), the GEF homology domain (residues 318-605), the glycine/acidic-rich tether (residues 752-764; also referred to as the gly/glu-rich linker), and the unique carboxy-terminal domain (residues 765-979);

FIG. 7 is a sequence alignment of sGNK, SEQ ID NO: 2, with a human homolog of Drosophila Bicaudal-D, SEQ ID NO: 18, and the human centrosomal protein C-Nap1, SEQ ID NO: 19;

FIG. 8 is an autoradiogram of an SDS-PAGE gel demonstrating that sGNK is phosphorylated by GNK, which is also autophosphorylated;

FIG. 9 is a chromatographic profile, silver-stained polyacrylamide gel and an autoradiogram depicting the copurification of GNK and sGNK by HPLC on a microbore MonoQ column.

FIG. 10 is a map of the GNK genomic locus encoding exons 1 and 2, the homologous recombinant vector, and the positive control vector used in constructing a GNK gene targeting vector.

FIG. 11 shows the vascularization of yolk sacs from GNK sufficient (GNK+/+,

FIG. 11A, top) and GNK deficient (GNK−/−,

FIG. 11B, bottom) fetuses.

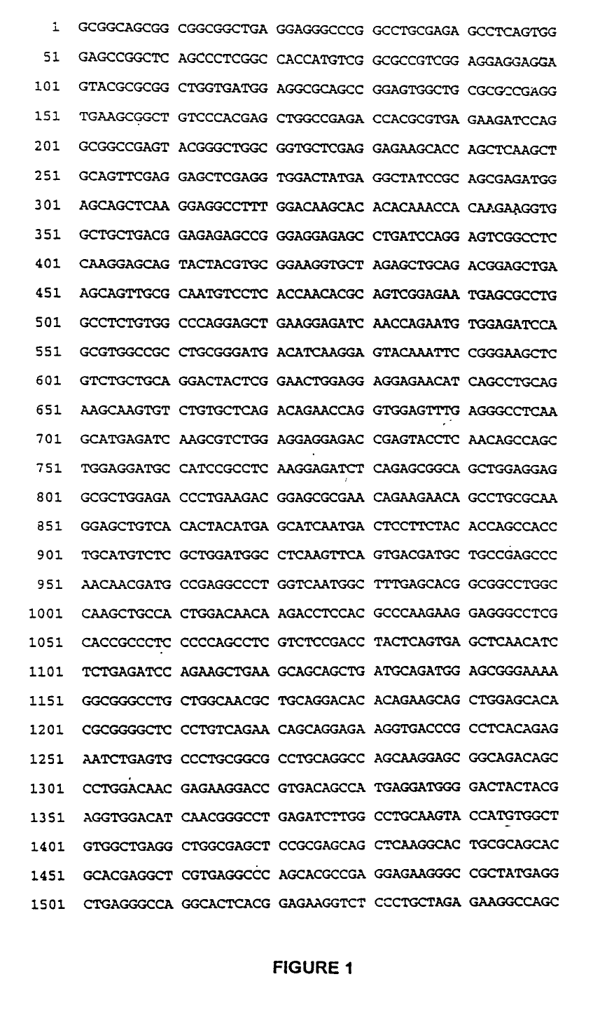

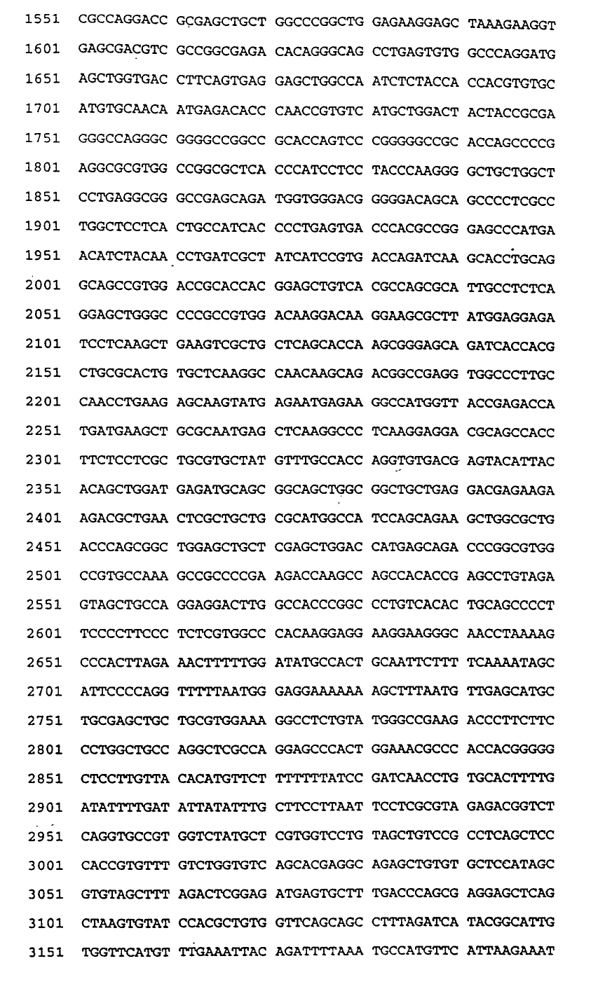

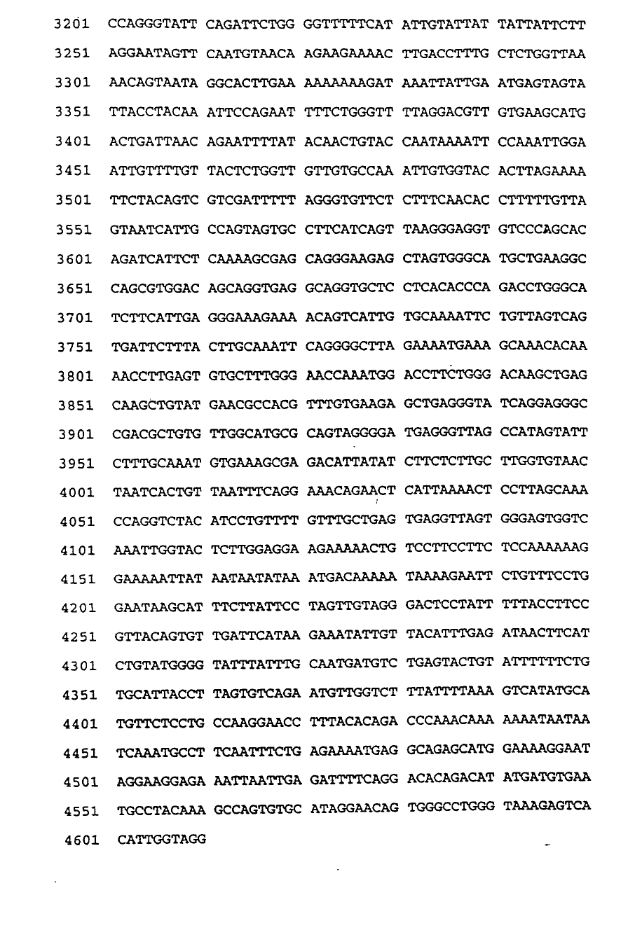

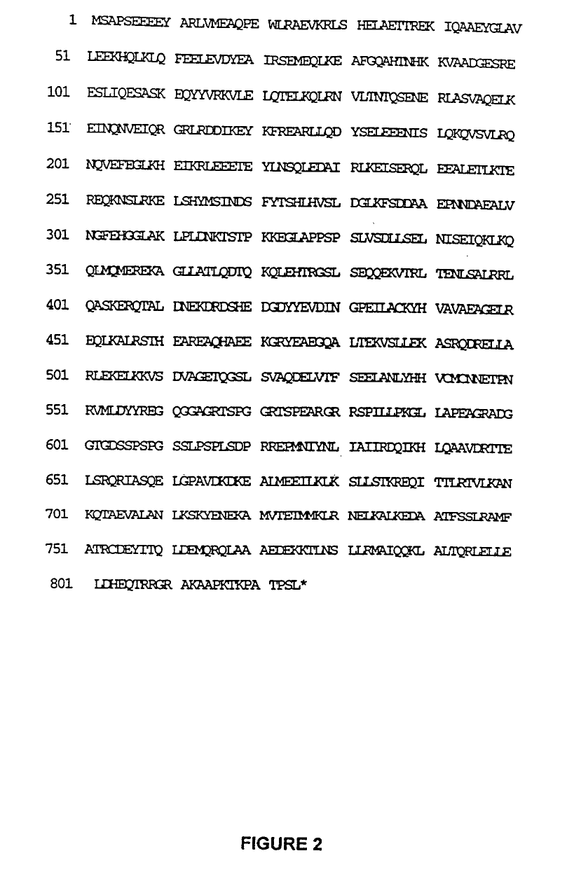

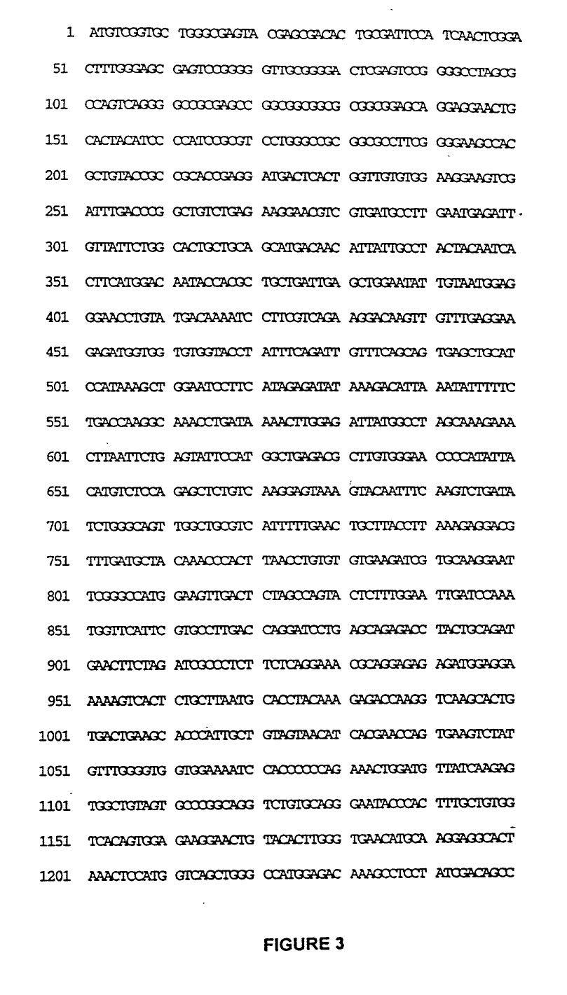

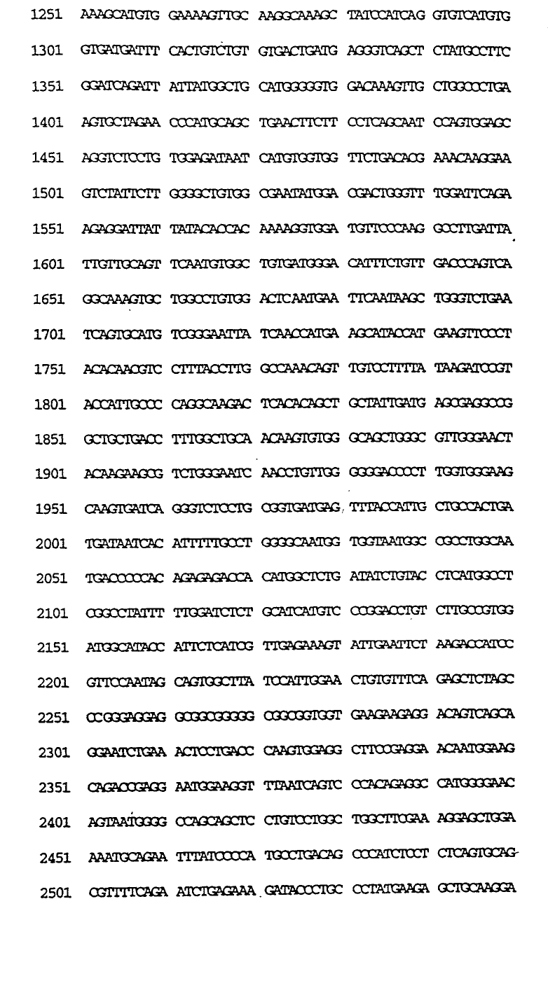

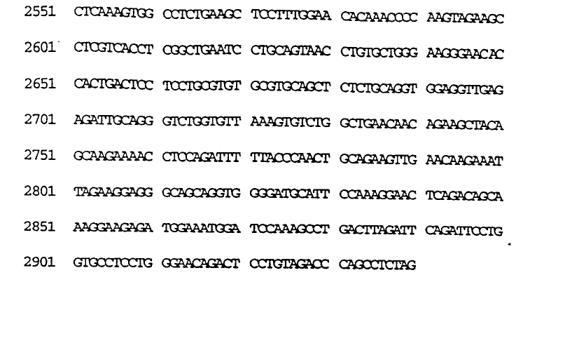

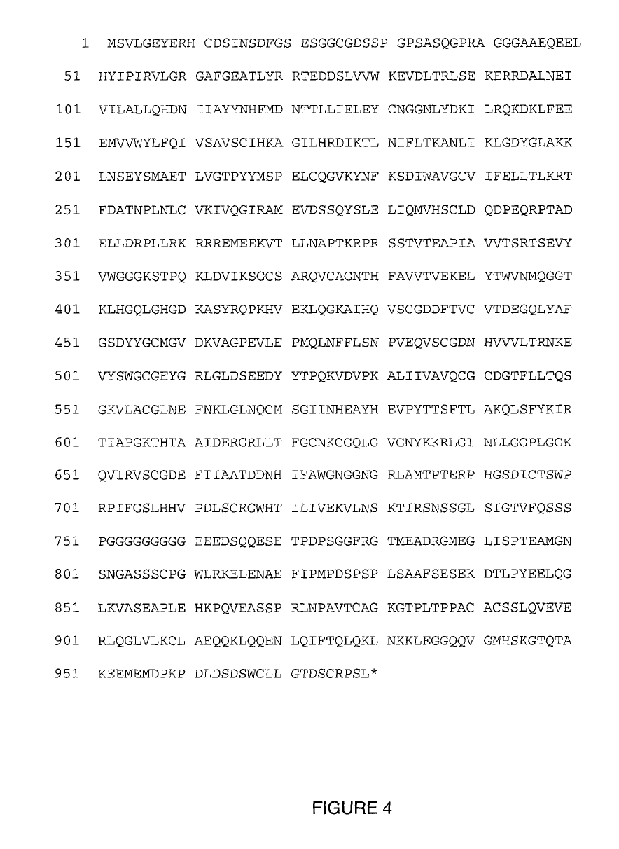

### DETAILED DESCRIPTION OF THE INVENTION

Throughout the specification various documents, including articles, books, patents, and patent applications, are cited. All of these documents are hereby incorporated by reference.

The nucleotide and amino acid sequence of GNK (identified therein as ITAK) were originally disclosed in U.S. application Ser. No. 08/870,529, which is herein incorporated by reference. Subsequent characterization studies by the instant inventors have identified a novel role for GNK in angiogenesis or neovascularization. Additionally, a second polypeptide, sGNK, which is a physiological substrate of and co-purifies with GNK, has been characterized and its nucleotide and amino acid sequence are disclosed. The sequences of GNK and sGNK were individually compared to non-redundant protein and nucleotide database sequences (National Ctr. For Biotechnol. Information (NCBI), Bethesda, Md.) using the BLAST algorithm (Altschul et al., J. Mol. Biol. 215:403, 1990).

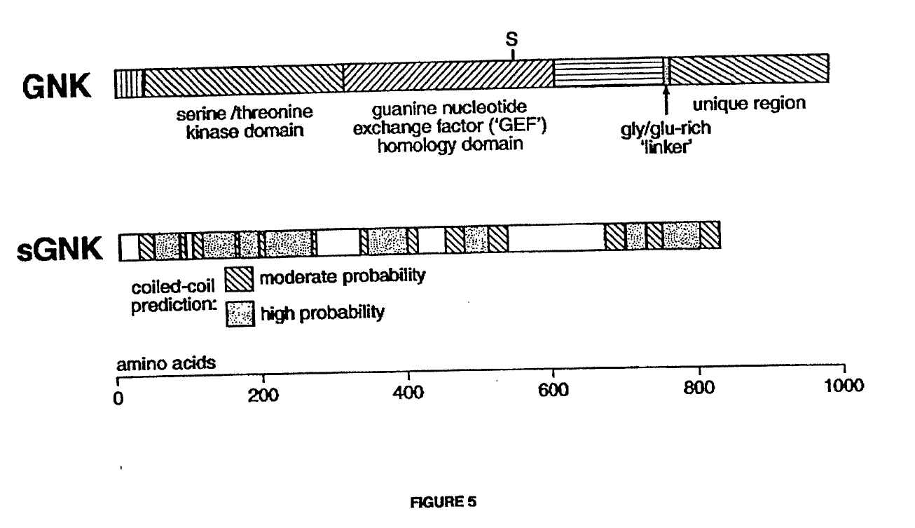

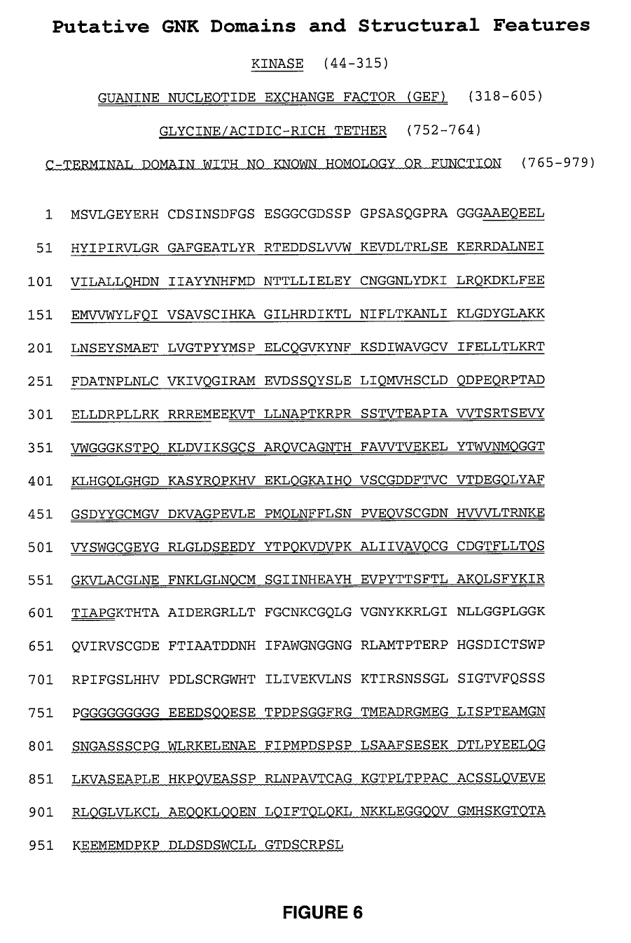

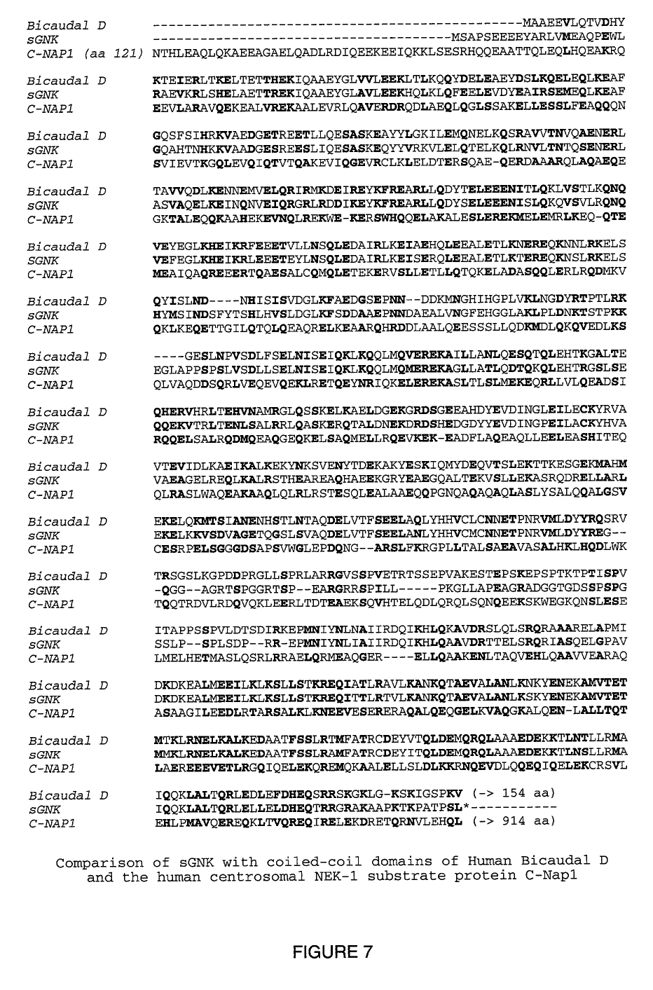

GNK was found to contain an N-terminal kinase domain, followed by a domain homologous to the Guanine nucleotide Exchange Factor (GEF) family of proteins, a short glycine/acidic-rich tether region and a C-terminal domain of unknown function with no significant homology to any known sequences. (See FIGS. 5 and 6). The sequence of sGNK, an approximately 90 kilodalton (kDa) protein predicted to have a high degree of coiled-coil structure, is similar to the Drosophila Bicaudal-D gene and has region of high homology with a newly discovered protein, C-Nap1. (See FIGS. 5 and 7).

GNK

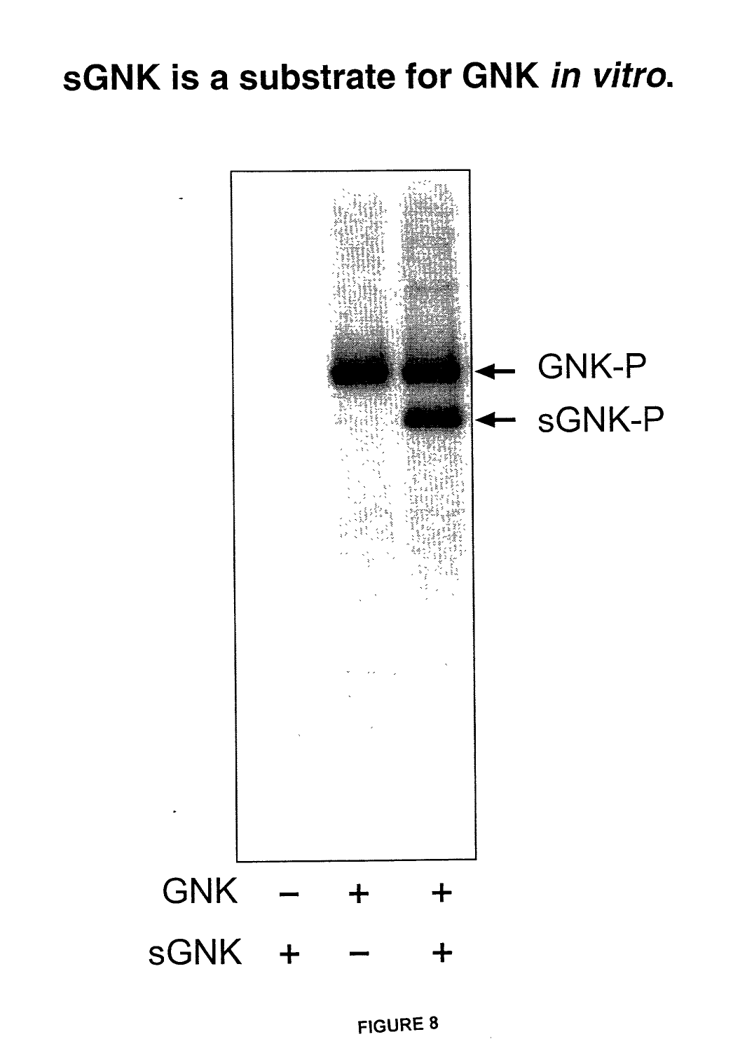

GNK is a protein kinase with an approximate molecular weight of 110 kDa, that will phosphorylate itself (autophosphorylation), sGNK, and possibly other undetermined physiological substrates under appropriate conditions. (See FIG. 8). Phosphorylated-GNK demonstrates a strong tendency to oligomerize. Based on SDS-PAGE and Superdex 200 gel filtration chromatography analyses, phosphorylated-GNK forms trimers and also higher-order complexes.

The kinase domain of GNK is most similar to the NIMA family of kinases, particularly Nek2 (NIMA-related kinase 2), a dual specificity kinase associated with regulation of the cell cycle. Nek2 associates with the centrosomes of all cells during all stages of the cell cycle and has been shown to be a bona fide component of the core centrosome. (Fry et al., EMBO J. 17:470, 1998). Overexpression of Nek2 results in splitting of the centrosome, dispersal of centrosomal material, and interference with microtubule regrowth, which profoundly affects centrosome structure and activity. (Id.). It has been proposed that Nek2 plays a role in severing the connection between the two duplicated centrosomes prior to the onset of mitosis by phosphorylating centrosomal “glue” proteins. (Id.).

The “GEF-like” domain of GNK is located between the kinase domain and the glycine/acidic-rich tether region. GEF proteins are activators of the Ras superfamily of proteins. (Overbeck et al., Mol. Repro. and Dev., 42:468, 1995). Members of the Ras superfamily are critical downstream components in signal transduction pathways that are initiated by the binding of extracellular ligands to transmembrane receptors possessing tyrosine kinase activity. Ras superfamily proteins are GTPases, which bind and hydrolyze GTP. They have been shown to regulate a wide variety of cellular activities, such as cell proliferation and differentiation, cytoskeletal organization, nuclear transport, and cell cycle regulation. Ras superfamily proteins are active when GTP is bound and inactive when GDP is bound. GEFs are positive regulators of Ras activity, promoting the release of bound GDP and facilitating GTP binding.

The glycine/acidic-rich tether is a series of nine consecutive glycine residues followed by three glutamic acid residues and an aspartic acid residue. This region of thirteen contiguous amino acid residues is located at position 752-764 of the GNK amino acid sequence. (See FIG. 6). This region is believed to serve as a molecular linker or separator that would effectively isolate the downstream novel C-terminal domain from the remainder of the molecule.

To further characterize GNK, cells were generated that lacked functional GNK. Attempts to develop GNK-deficient, or “knock-out”, mice by crossing GNK heterozygotes (GNK +/−) were unsuccessful as the homozygous phenotype was lethal. Viable GNK −/− fetuses were present at the expected frequency between e9.5-11.5, but were under-represented by e13.5. The GNK null fetuses were growth retarded, i.e., approximately 50% smaller than wild-type littermates by e13.5. The GNK-deficient fetuses also displayed reduced vascularization in the yolk sac and placenta, indicating that GNK plays a critical role in angiogenesis and vascular biology. GNK −/− murine fibroblasts were generated from viable e11.5-13.5 GNK −/− fetuses.

These data suggest that inhibitors of GNK may be useful in inhibiting vascularization (i.e., angiogenesis and vasculogenesis). Inhibitors of angiogenesis will be clinically beneficial in those cases where excessive blood vessel development is detrimental. For example, arresting vascularization may be useful in treating proliferative retinopathy, which can lead to vision loss in diabetics and premature infants. Angiogenesis inhibitors may arrest malignant tumor development at primary and secondary sites by reducing tumor vascularization. Additionally, angiogenesis inhibitors may be useful in limiting the development and spread of warts and benign tumors. In the absence of a blood supply, a tumor cannot grow beyond 1-2 mm in size. Such inhibitors may also be useful in treating other disorders associated with inappropriate blood vessel development, including arthritis and psoriasis.

Activators of GNK may be useful in stimulating blood vessel development in those cases where this might be advantageous, for example, during wound repair and cardiac dysfunction. GNK activators may also be useful in stimulating the revascularization of brain tissue following stroke.

sGNK

sGNK shows a high degree of sequence homology with the Bicaudal-D gene of Drosophila. The Bicaudal-D gene encodes a cytoskeleton-like coiled coil polypeptide with a leucine zipper and five a-helix domains. (Baens and Marynen, Genomics, 45:601, 1997). Mutations in bicaudal-D disrupt the cytoskeleton, interfere with messenger RNA (mRNA) sorting, and disrupt the polarity of the developing embryo. (Id.). A human homologue of bicaudal-D has recently been reported and there is evidence to suggest there may be additional human homologs. (Id.).

sGNK also contains a region that is highly homologous to C-Nap1, a novel centrosomal coiled coil protein that appears to be the substrate of Nek2. (Fry et al., J. Cell Biol. 141:1563, 1998). C-Nap1, like Nek2, is a core component of the human centrosome, that associates with centrosomes independently of microtubules. (Id.). C-Nap1 and Nek2 are known to co-localize in the centrosome and both have been detected in all cell types examined. (Id.). A recent model suggests that C-Nap1 may function as part of the centrosomal “glue”, by linking the ends of centrioles to each other during interphase. C-Nap1 is believed to be phosphorylated by Nek2 at the onset of mitosis, causing C-Nap1 to depolymerize or degrade which in turn permits the centrosomes to split during mitosis.

sGNK co-purifies with GNK, suggesting they may form a higher-order complex. sGNK is phosphorylated by GNK in vitro, suggesting an interaction similar to that seen with Nek2 and C-Nap1. Activators and inhibitors of sGNK may thus be useful in enhancing or decreasing angiogenesis. Inhibitors or sGNK phosphorylation will block subsequent biological activities of the protein, such as interfering with cell division or differentiation or blocking a signaling pathway. Activators of sGNK are expected to enhance its biological properties.

Nucleic Acid Molecules

In a particular embodiment, the invention relates to certain isolated nucleotide sequences that are free from contaminating endogenous material. A “nucleotide sequence” refers to a polynucleotide molecule in the form of a separate fragment or as a component of a larger nucleic acid construct. The nucleic acid molecule has been derived from DNA or RNA isolated at least once in substantially pure form and in a quantity or concentration enabling identification, manipulation, and recovery of its component nucleotide sequences by standard biochemical methods (such as those outlined in Sambrook et al., Molecular Cloning: A Laboratory Manual, 2nd sed., Cold Spring Harbor Laboratory, Cold Spring Harbor, N.Y., 1989). Such sequences are preferably provided and/or constructed in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region.

Nucleic acid molecules of the invention include DNA in both single-stranded and double-stranded form, as well as the RNA complement thereof. DNA includes, for example, cDNA, genomic DNA, chemically synthesized DNA, DNA amplified by PCR, and combinations thereof. The DNA molecules of the invention include full length genes as well as polynucleotides and fragments thereof. Genomic DNA may be isolated by conventional techniques, e.g., using the cDNA of SEQ ID NO:1, SEQ ID NO:3, or suitable fragments thereof, as a probe. The GNK and sGNK nucleic acids of the invention are preferentially derived from human sources, but the invention includes those derived from other mammalian species, as well.

Preferred Sequences

Particularly preferred nucleotide sequences of the invention are SEQ ID NO: 1 and SEQ ID NO: 3, which encode sGNK and GNK, respectively, as set forth above. A clone having the nucleotide sequence of SEQ ID NO: 1 was isolated as described in Example 2. The amino acid sequence encoded by the DNA of SEQ ID NO: 1 is shown in SEQ ID NO: 2. This sequence identifies sGNK, the physiological substrate of GNK. A clone having the nucleotide sequence of SEQ ID NO: 3 has also been isolated. The amino acid sequence encoded by the DNA of SEQ ID NO: 3 is shown in SEQ ID NO: 4. This sequence identifies GNK as a member of the protein kinase superfamily.

Additional Sequences

Due to the known degeneracy of the genetic code, wherein more than one codon can encode the same amino acid, a DNA sequence can vary from that shown in SEQ ID NO: 1 or SEQ ID NO: 3, and still encode a polypeptide having the amino acid sequence of SEQ ID NO: 2 or SEQ ID NO:4, respectively. Such variant DNA sequences can result from silent mutations (e.g., occurring during PCR amplification), or can be the product of deliberate mutagenesis of a native sequence.

The invention thus provides isolated DNA sequences encoding polypeptides of the invention, selected from: (a) DNA comprising the nucleotide sequence of SEQ ID NO: 1 or SEQ ID NO: 3; (b) DNA encoding the polypeptides of SEQ ID NO: 2 or SEQ ID NO: 4; (c) DNA capable of hybridizing with a DNA of (a) or (b) under conditions of moderate stringency and which encodes polypeptides of the invention; (d) DNA capable of hybridizing with a DNA of (a) or (b) under conditions of high stringency and which encodes polypeptides of the invention; and (e) DNA which is degenerate as a result of the genetic code to a DNA defined in (a), (b), (c), or (d) and which encode polypeptides of the invention. Of course, polypeptides encoded by such DNA sequences are encompassed by the invention.

As used herein, conditions of moderate stringency can be readily determined by those having ordinary skill in the art based on, for example, the length of the DNA. The basic conditions are set forth by Sambrook et al. Molecular Cloning: A Laboratory Manual, 2 ed. Vol. 1, pp. 1.101-104, Cold Spring Harbor Laboratory Press, (1989), and include use of a pre-washing solution for the nitrocellulose filters 5× SSC, 0.5% SDS, 1.0 mM EDTA (pH 8.0), hybridization conditions of about 50% formamide, 6× SSC at about 42° C. (or other similar hybridization solution, such as Stark's solution, in about 50% formamide at about 42° C.), and washing conditions of about 60° C., 0.5× SSC, 0.1% SDS. Conditions of high stringency can also be readily determined by the skilled artisan based on, for example, the length of the DNA. Generally, such conditions are defined as hybridization conditions as above, and with washing at approximately 68° C., 0.2× SSC, 0.1% SDS. The skilled artisan will recognize that the temperature and wash solution salt concentration can be adjusted as necessary according to factors such as the length of the probe.

Also included as an embodiment of the invention is DNA encoding polypeptide fragments and polypeptides comprising inactivated site(s) for myristoylation, palmitoylation, prenylation (supporting the thioether linkage of a farnesyl or geranylgeranyl moiety) or glycosyl phosphatidylinositol (GPI) linkage, inactivated protease processing site(s), or conservative amino acid substitution(s).

In another embodiment, the nucleic acid molecules of the invention also comprise nucleotide sequences that are at least 80% identical to a native sequence. Also contemplated are embodiments in which a nucleic acid molecule comprises a sequence that is at least 90% identical, at least 95% identical, at least 98% identical, at least 99% identical, or at least 99.9% identical to a native sequence.

The percent identity may be determined by visual inspection and mathematical calculation. Alternatively, the percent identity of two nucleic acid sequences can be determined by comparing sequence information using the GAP computer program, version 6.0 described by Devereux et al. (Nucl. Acids Res. 12:387, 1984) and available from the University of Wisconsin Genetics Computer Group (UWGCG). The preferred default parameters for the GAP program include: (1) a unary comparison matrix (containing a value of 1 for identities and 0 for non-identities) for nucleotides, and the weighted comparison matrix of Gribskov and Burgess, Nucl. Acids Res. 14:6745, 1986, as described by Schwartz and Dayhoff, eds., Atlas of Protein Sequence and Structure, National Biomedical Research Foundation, pp. 353-358, 1979; (2) a penalty of 3.0 for each gap and an additional 0.10 penalty for each symbol in each gap; and (3) no penalty for end gaps. Other programs used by persons skilled in the art of sequence comparison, such as those employing the BLAST algorithm, may also be employed.

The invention also provides isolated nucleic acids useful in the production of polypeptides. Such polypeptides may be prepared by any of a number of conventional techniques. The DNA sequence encoding the GNK or sGNK polypeptides, or desired fragments thereof, may be subcloned into an expression vector for production of the polypeptide or fragment. The DNA sequence advantageously is fused to a sequence encoding a suitable leader or signal peptide. Alternatively, the desired fragment may be chemically synthesized using known techniques. DNA fragments also may be produced by restriction endonuclease digestion of a full length cloned DNA sequence, and isolated by electrophoresis on agarose gels. If necessary, oligonucleotides that reconstruct the 5′ or 3′ terminus to a desired point may be ligated to a DNA fragment generated by restriction enzyme digestion. Such oligonucleotides may additionally contain a restriction endonuclease cleavage site upstream of the desired coding sequence, and position an initiation codon (ATG) at the N-terminus of the coding sequence.

The well-known polymerase chain reaction (PCR) procedure also may be employed to isolate and amplify a DNA sequence encoding a desired protein fragment. Oligonucleotides that define the desired termini of the DNA fragment are employed as 5′ and 3′ primers. The oligonucleotides may additionally contain recognition sites for restriction endonucleases, to facilitate insertion of the amplified DNA fragment into an expression vector. PCR techniques are described in Saiki et al., Science 239:487 (1988); Recombinant DNA Methodology, Wu et al., eds., Academic Press, Inc., San Diego (1989), pp. 189-196; and PCR Protocols: A Guide to Methods and Applications, Innis et al., eds., Academic Press, Inc. (1990).

Polypeptides and Fragments Thereof

The invention encompasses GNK and sGNK polypeptides and fragments thereof in various forms, including those that are naturally occurring or produced through various techniques such as procedures involving recombinant DNA technology. Such forms include, but are not limited to, derivatives, variants, and oligomers, as well as fusion proteins or fragments thereof.

Polypeptides and Fragments Thereof

The polypeptides of the invention include full length proteins encoded by the nucleic acid sequences set forth above. Polypeptide fragments of these nucleotide sequences are also intended to be within the scope of the invention. For example, a particular polypeptide fragment of GNK has been identified with kinase activity approximately 3-4 times higher than full length GNK. This particularly preferred fragment comprises amino acids 2 to 340 of SEQ ID NO: 4.

The polypeptide of SEQ ID NO: 4 includes an N-terminal leader region of 43 amino acids followed by a kinase domain comprising amino acids 44 to 315, a Guanine nucleotide Exchange Factor (GEF) homology region comprising amino acids 318 through 605, a short glycine/acidic-rich tether region comprising amino acids 752 through 764 and a novel C-terminal cytoplasmic domain having no significant homology with any sequences in the computer data bases, comprising amino acids 765 to 979. A spacer region comprises amino acids 606 to 751.

The skilled artisan will recognize that the above-described boundaries of such regions of the polypeptide are approximate and that the boundaries of the kinase domain (which may be predicted by using computer programs available for that purpose) may differ from those described above.

The polypeptides of the invention may be cytosolic or they may be genetically engineered to be secreted, i.e., capable of being secreted from the cells in which they are expressed. In general, secreted polypeptides may be identified (and distinguished from cytosolic counterparts) by separating intact cells which express the desired polypeptide from the culture medium, e.g., by centrifugation, and assaying the medium (supernatant) for the presence of the desired polypeptide. The presence of polypeptide in the medium indicates that the polypeptide was secreted from the cells.

In general, the use of soluble forms is advantageous for certain applications. Purification of such polypeptides from recombinant host cells is facilitated, since they are secreted from the cells. Further, secreted polypeptides may be preferable for therapeutic administration.

Also provided herein are polypeptide fragments comprising at least 20, or at least 30, contiguous amino acids of the sequence of SEQ ID NO: 2 or of SEQ ID NO: 4. Fragments derived from these amino acid sequences find use in studies of signal transduction, in regulating cellular processes associated with transduction of biological signals, and in vascular biology studies. Polypeptide fragments may also be employed as immunogens, in generating antibodies.

Variants

Naturally occurring variants as well as derived variants of the polypeptides and fragments are provided herein.

Variants may exhibit amino acid sequences that are at least 80% identical. Also contemplated are embodiments in which a polypeptide or fragments thereof comprises an amino acid sequence that is at least 90% identical, at least 95% identical, at least 98% identical, at least 99% identical, or at least 99.9% identical to the preferred polypeptide or fragment thereof. Percent identity may be determined by visual inspection and mathematical calculation. Alternatively, the percent identity of two protein sequences can be determined by comparing sequence information using the GAP computer program, based on the algorithm of Needleman and Wunsch (J. Mol. Bio. 48:443, 1970) and available from the University of Wisconsin Genetics Computer Group (UWGCG). The preferred default parameters for the GAP program include: (1) a scoring matrix, blosum62, as described by Henikoff and Henikoff (Proc. Natl. Acad. Sci. USA 89:10915, 1992); (2) a gap weight of 12; (3) a gap length weight of 4; and (4) no penalty for end gaps. Other programs used by one skilled in the art of sequence comparison may also be used.

The variants of the invention include, for example, those that result from alternate mRNA splicing events or from proteolytic cleavage. Alternate splicing of mRNA may, for example, yield a truncated but biologically active protein, such as a naturally occurring soluble form of the protein or a variant lacking a regulatory component. Variations attributable to proteolysis include, for example, differences in the N- or C-termini upon expression in different types of host cells, due to proteolytic removal of one or more terminal amino acids from the protein (generally from 1-5 terminal amino acids). Proteins in which differences in amino acid sequence are attributable to genetic polymorphism (allelic variation among individuals producing the protein) are also contemplated herein.

Additional variants within the scope of the invention include polypeptides that may be modified to create derivatives thereof by forming covalent or aggregative conjugates with other chemical moieties, such as glycosyl groups, lipids, phosphate, acetyl groups and the like. Covalent derivatives may be prepared by linking the chemical moieties to functional groups on amino acid side chains or at the N-terminus or C-terminus of a polypeptide. Conjugates comprising diagnostic (detectable) or therapeutic agents attached thereto are contemplated herein, as discussed in more detail below.

Other derivatives include covalent or aggregative conjugates of the polypeptides with other proteins or polypeptides, such as by synthesis in recombinant culture as N-terminal or C-terminal fusions. Examples of fusion proteins are discussed below in connection with oligomers. Further, fusion proteins can comprise peptides added to facilitate purification and identification. Such peptides include, for example, poly-His or the antigenic identification peptides described in U.S. Pat. No. 5,011,912 and in Hopp et al., Bio/Technology 6:1204, 1988. One such peptide is the FLAG® peptide, Asp-Tyr-Lys-Asp-Asp-Asp-Asp-Lys, which is highly antigenic and provides an epitope reversibly bound by a specific monoclonal antibody, enabling rapid assay and facile purification of expressed recombinant protein. A murine hybridoma designated 4E11 produces a monoclonal antibody that binds the FLAG® peptide in the presence of certain divalent metal cations, as described in U.S. Pat. No. 5,011,912, hereby incorporated by reference. The 4E11 hybridoma cell line has been deposited with the American Type Culture Collection under accession no. HB 9259. Monoclonal antibodies that bind the FLAG® peptide are available from Eastman Kodak Co., Scientific Imaging Systems Division, New Haven, Conn.

Among the variant polypeptides provided herein are variants of native polypeptides that retain the native biological activity or the substantial equivalent thereof. One example is a variant that binds with essentially the same binding affinity as does the native form. Binding affinity can be measured by conventional procedures, e.g., as described in U.S. Pat. No. 5,512,457 and as set forth below.

Variants include polypeptides that are substantially homologous to the native form, but which have an amino acid sequence different from that of the native form because of one or more deletions, insertions or substitutions. Particular embodiments include, but are not limited to, polypeptides that comprise from one to ten deletions, insertions or substitutions of amino acid residues, when compared to a native sequence.

A given amino acid may be replaced, for example, by a residue having similar physiochemical characteristics. Examples of such conservative substitutions include substitution of one aliphatic residue for another, such as Ile, Val, Leu, or Ala for one another; substitutions of one polar residue for another, such as between Lys and Arg, Glu and Asp, or Gln and Asn; or substitutions of one aromatic residue for another, such as Phe, Trp, or Tyr for one another. Other conservative substitutions, e.g., involving substitutions of entire regions having similar hydrophobicity characteristics, are well known.

Similarly, the DNAs of the invention include variants that differ from a native DNA sequence because of one or more deletions, insertions or substitutions, but that encode a biologically active polypeptide.

The invention further includes polypeptides of the invention with or without associated native-pattern lipid anchors, e.g., myristoylation, palmitoylation, prenylation, etc. Post-translational modifications such as the enzyme-catalyzed addition of myristic or palmitic acid residues or GPI anchors cause the modified protein to become membrane-bound.

Correspondingly, similar DNA constructs that encode various additions or substitutions of amino acid residues or sequences, or deletions of terminal or internal residues or sequences are encompassed by the invention. For example, myristylation addition sites in the polypeptide can be modified to preclude myristylation, allowing expression of a fatty acid-free analog in mammalian and yeast expression systems.

In another example of variants, sequences encoding Cys residues that are not essential for biological activity can be altered to cause the Cys residues to be deleted or replaced with other amino acids, preventing formation of incorrect intramolecular disulfide bridges upon folding or renaturation.

Other variants are prepared by modification of adjacent dibasic amino acid residues, to enhance expression in yeast systems in which KEX2 protease activity is present. EP 212,914 discloses the use of site-specific mutagenesis to inactivate KEX2 protease processing sites in a protein. KEX2 protease processing sites are inactivated by deleting, adding or substituting residues to alter Arg-Arg, Arg-Lys, and Lys-Arg pairs to eliminate the occurrence of these adjacent basic residues. Lys-Lys pairings are considerably less susceptible to KEX2 cleavage, and conversion of Arg-Lys or Lys-Arg to Lys-Lys represents a conservative and preferred approach to inactivating KEX2 sites.

Oligomers

Encompassed by the invention homo- and hetero-oligomers of GNK and/or sGNK, or fusion proteins that contain GNK and/or sGNK polypeptides. Such oligomers may be in the form of covalently-linked or non-covalently-linked multimers, including dimers, trimers, or higher oligomers. In one aspect of the invention, the oligomers maintain the binding ability of the polypeptide components and provide binding sites that are bivalent, trivalent, etc.

One embodiment of the invention is directed to oligomers comprising multiple polypeptides joined via covalent or non-covalent interactions between peptide moieties fused to the polypeptides. Such peptides may be peptide linkers (spacers), or peptides that have the property of promoting oligomerization. Leucine zippers and certain polypeptides derived from antibodies are among the peptides that can promote oligomerization of the polypeptides attached thereto, as described in more detail below.

Immunoglobulin-based Oligomers

As one alternative, an oligomer is prepared using polypeptides derived from immunoglobulins. Preparation of fusion proteins comprising certain heterologous polypeptides fused to various portions of antibody-derived polypeptides (including the Fc domain) has been described, e.g., by Ashkenazi et al. (Proc. Natl. Acad. Sci. USA 88:10535, 1991); Byrn et al. (Nature 344:677, 1990); and Hollenbaugh and Aruffo (“Construction of Immunoglobulin Fusion Proteins”, in Current Protocols in Immunology, Suppl. 4, pages 10.19.1-10.19.11, 1992).

One embodiment of the present invention is directed to a dimer comprising two fusion proteins created by fusing a polypeptide of the invention to an Fc polypeptide derived from an antibody. A gene fusion encoding the polypeptide/Fc fusion protein is inserted into an appropriate expression vector. Polypeptide/Fc fusion proteins are expressed in host cells transformed with the recombinant expression vector, and allowed to assemble much like antibody molecules, whereupon interchain disulfide bonds form between the Fc moieties to yield divalent molecules.

The term “Fc polypeptide” as used herein includes native and mutein forms of polypeptides made up of the Fc region of an antibody comprising any or all of the CH domains of the Fc region. Truncated forms of such polypeptides containing the hinge region that promotes dimerization are also included. Preferred polypeptides comprise an Fc polypeptide derived from a human IgG1 antibody.

One suitable Fc polypeptide, described in PCT application WO 93/10151 (hereby incorporated by reference), is a single chain polypeptide extending from the N-terminal hinge region to the native C-terminus of the Fc region of a human IgG1 antibody. Another useful Fc polypeptide is the Fc mutein described in U.S. Pat. No. 5,457,035 and in Baum et al., (EMBO J. 13:3992-4001, 1994) incorporated herein by reference. The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors.

The above-described fusion proteins comprising Fc moieties (and oligomers formed therefrom) offer the advantage of facile purification by affinity chromatography using Protein A or Protein G columns.

Peptide-linker Based Oligomers

Alternatively, the oligomer is a fusion protein comprising multiple polypeptides, with or without peptide linkers (spacer peptides). Among the suitable peptide linkers are those described in U.S. Pat. Nos. 4,751,180 and 4,935,233, which are hereby incorporated by reference. A DNA sequence encoding a desired peptide linker may be inserted between, and in the same reading frame as, the DNA sequences of the invention, using any suitable conventional technique. For example, a chemically synthesized oligonucleotide encoding the linker may be ligated between the sequences. In particular embodiments, a fusion protein comprises from two to four soluble GNK and/or sGNK polypeptides, separated by peptide linkers.

Leucine-zippers

Another method for preparing the oligomers of the invention involves use of a leucine zipper. Leucine zipper domains are peptides that promote oligomerization of the proteins in which they are found. Leucine zippers were originally identified in several DNA-binding proteins (Landschulz et al., Science 240:1759, 1988), and have since been found in a variety of different proteins. Among the known leucine zippers are naturally occurring peptides and derivatives thereof that dimerize or trimerize.

The zipper domain (also referred to herein as an oligomerizing, or oligomer-forming, domain) comprises a repetitive heptad repeat, often with four or five leucine residues interspersed with other amino acids. Examples of zipper domains are those found in the yeast transcription factor GCN4 and a heat-stable DNA-binding protein found in rat liver (C/EBP; Landschulz et al., Science 243:1681, 1989). Two nuclear transforming proteins, fos and jun, also exhibit zipper domains, as does the gene product of the murine proto-oncogene, c-myc (Landschulz et al., Science 240:1759, 1988). The products of the nuclear oncogenes fos and jun comprise zipper domains that preferentially form heterodimer (O'Shea et al., Science 245:646, 1989, Turner and Tjian, Science 243:1689, 1989). The zipper domain is necessary for biological activity (DNA binding) in these proteins.

The fusogenic proteins of several different viruses, including paramyxovirus, coronavirus, measles virus and many retroviruses, also possess zipper domains (Buckland and Wild, Nature 338:547,1989; Britton, Nature 353:394, 1991; Delwart and Mosialos, AIDS Research and Human Retroviruses 6:703, 1990). The zipper domains in these fusogenic viral proteins are near the transmembrane region of the proteins; it has been suggested that the zipper domains could contribute to the oligomeric structure of the fusogenic proteins. Oligomerization of fusogenic viral proteins is involved in fusion pore formation (Spruce et al, Proc. Natl. Acad. Sci. U.S.A. 88:3523, 1991). Zipper domains have also been recently reported to play a role in oligomerization of heat-shock transcription factors (Rabindran et al., Science 259:230, 1993).

Zipper domains fold as short, parallel coiled coils (O'Shea et al., Science 254:539; 1991). The general architecture of the parallel coiled coil has been well characterized, with a “knobs-into-holes” packing as proposed by Crick in 1953 (Acta Crystallogr. 6:689). The dimer formed by a zipper domain is stabilized by the heptad repeat, designated (abcdefg)ₙ according to the notation of McLachlan and Stewart (J. Mol. Biol. 98:293; 1975), in which residues a and d are generally hydrophobic residues, with d being a leucine, which line up on the same face of a helix. Oppositely-charged residues commonly occur at positions g and e. Thus, in a parallel coiled coil formed from two helical zipper domains, the “knobs” formed by the hydrophobic side chains of the first helix are packed into the “holes” formed between the side chains of the second helix.

The residues at position d (often leucine) contribute large hydrophobic stabilization energies, and are important for oligomer formation (Krystek: et al., Int. J. Peptide Res. 38:229, 1991). Lovejoy et al. (Science 259:1288, 1993) recently reported the synthesis of a triple-stranded a-helical bundle in which the helices run up-up-down. Their studies confirmed that hydrophobic stabilization energy provides the main driving force for the formation of coiled coils from helical monomers. These studies also indicate that electrostatic interactions contribute to the stoichiometry and geometry of coiled coils. Further discussion of the structure of leucine zippers is found in Harbury et al. (Science 262:1401, 26 November 1993).

Examples of leucine zipper domains suitable for producing soluble oligomeric proteins are described in PCT application WO 94/10308, and the leucine zipper derived from lung surfactant protein D (SPD) described in Hoppe et al. (FEBS Letters 344:191, 1994), hereby incorporated by reference. The use of a modified leucine zipper that allows for stable trimerization of a heterologous protein fused thereto is described in Fanslow et al. (Semin. Immunol. 6:267-278, 1994). Recombinant fusion proteins comprising a soluble polypeptide fused to a leucine zipper peptide are expressed in suitable host cells, and the soluble oligomer that forms is recovered from the culture supernatant.

Certain leucine zipper moieties preferentially form trimers. One example is a leucine zipper derived from lung surfactant protein D (SPD), as described in Hoppe et al. (FEBS Letters 344:191, 1994) and in U.S. Pat. No. 5,716,805, hereby incorporated by reference in their entirety. This lung SPD-derived leucine zipper peptide comprises the amino acid sequence Pro Asp Val Ala Ser Leu Arg Gln Gln Val Glu Ala Leu Gln Gly Gln Val Gln His Leu Gln Ala Ala Phe Ser Gln Tyr.

Another example of a leucine zipper that promotes trimerization is a peptide comprising the amino acid sequence Arg Met Lys Gln Ile Glu Asp Lys Ile Glu Glu Ile Leu Ser Lys Ile Tyr His Ile Glu Asn Glu Ile Ala Arg Ile Lys Lys Leu Ile Gly Glu Arg, as described in U.S. Pat. No. 5,716,805. In one alternative embodiment, an N-terminal Asp residue is added; in another, the peptide lacks the N-terminal Arg residue.

Fragments of the foregoing zipper peptides that retain the property of promoting oligomerization may be employed as well. Examples of such fragments include, but are not limited to, peptides lacking one or two of the N-terminal or C-terminal residues presented in the foregoing amino acid sequences. Leucine zippers may be derived from naturally occurring leucine zipper peptides, e.g., via conservative substitution(s) in the native amino acid sequence, wherein the peptide's ability to promote oligomerization is retained.

Other peptides derived from naturally occurring trimeric proteins may be employed in preparing trimeric GNK or sGNK preparations. Alternatively, synthetic peptides that promote oligomerization may be employed. In particular embodiments, leucine residues in a leucine zipper moiety are replaced by isoleucine residues. Such peptides comprising isoleucine may be referred to as isoleucine zippers, but are encompassed by the term “leucine zippers” as employed herein.

Production of Polypeptides and Fragments Thereof

Expression, isolation and purification of the polypeptides and fragments of the invention may be accomplished by any suitable technique, including but not limited to the following:

Expression Systems

The present invention also provides recombinant cloning and expression vectors containing DNA, as well as host cells containing the recombinant vectors. Expression vectors comprising DNA may be used to prepare the polypeptides or fragments of the invention encoded by the DNA. A method for producing polypeptides comprises culturing host cells transformed with a recombinant expression vector encoding the polypeptide, under conditions that promote expression of the polypeptide, then recovering the expressed polypeptides from the culture. The skilled artisan will recognize that the procedure for purifying the expressed polypeptides will vary according to such factors as the type of host cells employed, and whether the polypeptide is membrane-bound or in a soluble, secreted form.

Any suitable expression system may be employed. The vectors include a DNA encoding a polypeptide or fragment of the invention, operably linked to suitable transcriptional or translational regulatory nucleotide sequences, such as those derived from a mammalian, microbial, viral, or insect gene. Examples of regulatory sequences include transcriptional promoters, operators, or enhancers, an mRNA ribosomal binding site, and appropriate sequences which control transcription and translation initiation and termination. Nucleotide sequences are operably linked when the regulatory sequence functionally relates to the DNA sequence. Thus, a promoter nucleotide sequence is operably linked to a DNA sequence if the promoter nucleotide sequence controls the transcription of the DNA sequence. An origin of replication that confers the ability to replicate in the desired host cells, and a selection gene by which transformants are identified, are generally incorporated into the expression vector.

In addition, a sequence encoding an appropriate signal peptide (native or heterologous) can be incorporated into expression vectors. A DNA sequence for a signal peptide (secretory leader) may be fused in frame to the nucleic acid sequence of the invention so that the DNA is initially transcribed, and the mRNA translated, into a fusion protein comprising the signal peptide. A signal peptide that is functional in the intended host cells promotes extracellular secretion of the polypeptide. The signal peptide is cleaved from the polypeptide upon secretion of polypeptide from the cell.

The skilled artisan will also recognize that the position(s) at which the signal peptide is cleaved may differ from that predicted by computer program, and may vary according to such factors as the type of host cells employed in expressing a recombinant polypeptide. A protein preparation may include a mixture of protein molecules having different N-terminal amino acids, resulting from cleavage of the signal peptide at more than one site.

Suitable host cells for expression of polypeptides include prokaryotes, yeast or higher eukaryotic cells. Mammalian or insect cells are generally preferred for use as host cells. Appropriate cloning and expression vectors for use with bacterial, fungal, yeast, and mammalian cellular hosts are described, for example, in Pouwels et al. Cloning Vectors: A Laboratory Manual, Elsevier, N.Y., (1985). Cell-free translation systems could also be employed to produce polypeptides using RNAs derived from DNA constructs disclosed herein.

Prokaryotic Systems

Prokaryotes include gram-negative or gram-positive organisms. Suitable prokaryotic host cells for transformation include, for example, E. coli, Bacillus subtilis, Salmonella typhimurium, and various other species within the genera Pseudomonas, Streptomyces, and Staphylococcus. In a prokaryotic host cell, such as E. coli, a polypeptide may include an N-terminal methionine residue to facilitate expression of the recombinant polypeptide in the prokaryotic host cell. The N-terminal Met may be cleaved from the expressed recombinant polypeptide.

Expression vectors for use in prokaryotic host cells generally comprise one or more phenotypic selectable marker genes. A phenotypic selectable marker gene is, for example, a gene encoding a protein that confers antibiotic resistance or that supplies an autotrophic requirement. Examples of useful expression vectors for prokaryotic host cells include those derived from commercially available plasmids such as the cloning vector pBR322 (ATCC 37017). pBR322 contains genes for ampicillin and tetracycline resistance and thus provides simple means for identifying transformed cells. An appropriate promoter and a DNA sequence are inserted into the pBR322 vector. Other commercially available vectors include, for example, pKK223-3 (Pharmacia Fine Chemicals, Uppsala, Sweden) and pGEM1 (Promega Biotec, Madison, Wis., USA).

Promoter sequences commonly used for recombinant prokaryotic host cell expression vectors include β-lactamase (penicillinase), lactose promoter system (Chang et al., Nature 275:615, 1978; and Goeddel et al., Nature 281:544, 1979), tryptophan (trp) promoter system (Goeddel et al., Nucl. Acids Res. 8:4057, 1980; and EP-A-36776) and tac promoter (Maniatis, Molecular Cloning: A Laboratory Manual, Cold Spring Harbor Laboratory, p. 412, 1982). A particularly useful prokaryotic host cell expression system employs a phage λP_{L} promoter and a cI857ts thermolabile repressor sequence. Plasmid vectors available from the American Type Culture Collection which incorporate derivatives of the λP_{L} promoter include plasmid pHUB2 (resident in E. coli strain JMB9, ATCC 37092) and pPLc28 (resident in E. coli RR1, ATCC 53082).

Yeast Systems

Alternatively, the polypeptides may be expressed in yeast host cells, preferably from the Saccharomyces genus (e.g., S. cerevisiae). Other genera of yeast, such as Pichia or Kluyveromyces, may also be employed. Yeast vectors will often contain an origin of replication sequence from a 2μ yeast plasmid, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman et al., J. Biol. Chem. 255:2073, 1980) or other glycolytic enzymes (Hess et al., J. Adv. Enzyme Reg. 7:149, 1968; and Holland et al., Biochem. 17:4900, 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phospho-glucose isomerase, and glucokinase. Other suitable vectors and promoters for use in yeast expression are further described in Hitzeman, EPA-73,657. Another alternative is the glucose-repressible ADH2 promoter described by Russell et al. (J. Biol. Chem. 258:2674, 1982) and Beier et al. (Nature 300:724, 1982). Shuttle vectors replicable in both yeast and E. coli may be constructed by inserting DNA sequences from pBR322 for selection and replication in E. coli (Amp^{r} gene and origin of replication) into the above-described yeast vectors.

The yeast α-factor leader sequence may be employed to direct secretion of the polypeptide. The α-factor leader sequence is often inserted between the promoter sequence and the structural gene sequence. See, e.g., Kurjan et al., Cell 30:933, 1982 and Bitter et al., Proc. Natl. Acad. Sci. USA 81:5330, 1984. Other leader sequences suitable for facilitating secretion of recombinant polypeptides from yeast hosts are known to those of skill in the art. A leader sequence may be modified near its 3′ end to contain one or more restriction sites. This will facilitate fusion of the leader sequence to the structural gene.

Yeast transformation protocols are known to those of skill in the art. One such protocol is described by Hinnen et al., Proc. Natl. Acad. Sci. USA 75:1929, 1978. The Hinnen et al. protocol selects for Trp⁺ transformants in a selective medium, wherein the selective medium consists of 0.67% yeast nitrogen base, 0.5% casamino acids, 2% glucose, 10 mg/ml adenine and 20 mg/ml uracil.

Yeast host cells transformed by vectors containing an ADH2 promoter sequence may be grown for inducing expression in a “rich” medium. An example of a rich medium is one consisting of 1% yeast extract, 2% peptone, and 1% glucose supplemented with 80 mg/ml adenine and 80 mg/ml uracil. Derepression of the ADH2 promoter occurs when glucose is exhausted from the medium.

Mammalian or Insect Systems

Mammalian or insect host cell culture systems also may be employed to express recombinant polypeptides. Baculovirus systems for production of heterologous proteins in insect cells are reviewed by Luckow and Summers, Bio/Technology 6:47 (1988). Established cell lines of mammalian origin also may be employed. Examples of suitable mammalian host cell lines include the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells, HeLa cells, and BHK (ATCC CRL 10) cell lines, and the CV1/EBNA cell line derived from the African green monkey kidney cell line CV1 (ATCC CCL 70) as described by McMahan et al. (EMBO J. 10: 2821, 1991).

Established methods for introducing DNA into mammalian cells have been described (Kaufman, R. J., Large Scale Mammalian Cell Culture, 1990, pp. 15-69). Additional protocols using commercially available reagents, such as Lipofectamine lipid reagent (Gibco/BRL) or Lipofectamine-Plus lipid reagent, can be used to transfect cells (Felgner et al., Proc. Natl. Acad. Sci. USA 84:7413-7417, 1987). In addition, electroporation can be used to transfect mammalian cells using conventional procedures, such as those in Sambrook et al. (Molecular Cloning: A Laboratory Manual, 2 ed. Vol. 1-3, Cold Spring Harbor Laboratory Press, 1989). Selection of stable transformants can be performed using methods known in the art, such as, for example, resistance to cytotoxic drugs. Kaufman et al., Meth. in Enzymology 185:487-511, 1990, describes several selection schemes, such as dihydrofolate reductase (DHFR) resistance. A suitable host strain for DHFR selection can be CHO strain DX-B11, which is deficient in DHFR (Urlaub and Chasin, Proc. Natl. Acad. Sci. USA 77:4216-4220, 1980). A plasmid expressing the DHFR cDNA can be introduced into strain DX-B11, and only cells that contain the plasmid can grow in the appropriate selective media. Other examples of selectable markers that can be incorporated into an expression vector include cDNAs conferring resistance to antibiotics, such as G418 and hygromycin B. Cells harboring the vector can be selected on the basis of resistance to these compounds.

Transcriptional and translational control sequences for mammalian host cell expression vectors can be excised from viral genomes. Commonly used promoter sequences and enhancer sequences are derived from polyoma virus, adenovirus 2, simian virus 40 (SV40), and human cytomegalovirus. DNA sequences derived from the SV40 viral genome, for example, SV40 origin, early and late promoter, enhancer, splice, and polyadenylation sites can be used to provide other genetic elements for expression of a structural gene sequence in a mammalian host cell. Viral early and late promoters are particularly useful because both are easily obtained from a viral genome as a fragment, which can also contain a viral origin of replication (Fiers et al., Nature 273:113, 1978; Kaufman, Meth. in Enzymology, 1990). Smaller or larger SV40 fragments can also be used, provided the approximately 250 bp sequence extending from the Hind m site toward the Bgl I site located in the SV40 viral origin of replication site is included.

Additional control sequences shown to improve expression of heterologous genes from mammalian expression vectors include such elements as the expression augmenting sequence element (EASE) derived from CHO cells (Morris et al., Animal Cell Technology, 1997, pp. 529-534 and PCT Application WO 97/25420) and the tripartite leader (TPL) and VA gene RNAs from Adenovirus 2 (Gingeras et al., J. Biol. Chem. 257:13475-13491, 1982). The internal ribosome entry site (IRES) sequences of viral origin allows dicistronic mRNAs to be translated efficiently (Oh and Sarnow, Current Opinion in Genetics and Development 3:295-300, 1993; Ramesh et al., Nucleic Acids Research 24:2697-2700, 1996). Expression of a heterologous cDNA as part of a dicistronic mRNA followed by the gene for a selectable marker (e.g. DHFR) has been shown to improve transfectability of the host and expression of the heterologous cDNA (Kaufman, Meth. in Enzymology, 1990). Exemplary expression vectors that employ dicistronic mRNAs are pTR-DC/GFP described by Mosser et al., Biotechniques 22:150-161, 1997, and p2A5I described by Morris et al., Animal Cell Technology, 1997, pp. 529-534.

A useful high expression vector, pCAVNOT, has been described by Mosley et al., Cell 59:335-348, 1989. Other expression vectors for use in mammalian host cells can be constructed as disclosed by Okayama and Berg (Mol. Cell. Biol. 3:280, 1983). A useful system for stable high level expression of mammalian cDNAs in C127 murine mammary epithelial cells can be constructed substantially as described by Cosman et al. (Mol. Immunol. 23:935, 1986). A useful high expression vector, PMLSV N1/N4, described by Cosman et al., Nature 312:768, 1984, has been deposited as ATCC 39890. Additional useful mammalian expression vectors are described in EP-A-0367566, and in WO 91/18982, incorporated by reference herein. In yet another alternative, the vectors can be derived from retroviruses.

Another useful expression vector, pFLAG®, can be used. FLAG® technology is centered on the fusion of a low molecular weight (1 kD), hydrophilic, FLAG® marker peptide to the N-terminus of a recombinant protein expressed by pFLAG® expression vectors.

Regarding signal peptides that may be employed, the native signal peptide may be replaced by a heterologous signal peptide or leader sequence, if desired. The choice of signal peptide or leader may depend on factors such as the type of host cells in which the recombinant polypeptide is to be produced. To illustrate, examples of heterologous signal peptides that are functional in mammalian host cells include the signal sequence for interleukin-7 (IL-7) described in U.S. Pat. No. 4,965,195; the signal sequence for interleukin-2 receptor described in Cosman et al., Nature 312:768 (1984); the interleukin-4 receptor signal peptide described in EP 367,566; the type I interleukin-1 receptor signal peptide described in U.S. Pat. No. 4,968,607; and the type II interleukin-1 receptor signal peptide described in EP 460,846.

Purification

The invention also includes methods of isolating and purifying the polypeptides and fragments thereof.

Isolation and Purification

The “isolated” polypeptides or fragments thereof encompassed by this invention are polypeptides or fragments that are found in an environment that is not identical to their natural environment. The “purified” polypeptides or fragments thereof encompassed by this invention are essentially free of association with other proteins or polypeptides, for example, as a purification product of recombinant expression systems such as those described above or as a purified product from a non-recombinant source such as naturally occurring cells and/or tissues.

In one preferred embodiment, the purification of recombinant polypeptides or fragments can be accomplished using fusions of polypeptides or fragments of the invention to another polypeptide to aid in the purification of polypeptides or fragments of the invention. Such fusion partners can include the poly-His or other antigenic identification peptides described above as well as the Fc moieties described previously.

With respect to any type of host cell, as is known to the skilled artisan, procedures for purifying a recombinant polypeptide or fragment will vary according to such factors as the type of host cells employed and whether or not the recombinant polypeptide or fragment is secreted into the culture medium.

In general, the recombinant polypeptide or fragment can be isolated from the host cells if not secreted, or from the medium or supernatant if soluble and secreted, followed by one or more concentration, salting-out, ion exchange, hydrophobic interaction, affinity purification or size exclusion chromatography steps. As to specific ways to accomplish these steps, the culture medium first can be concentrated using a commercially available protein concentration filter, for example, an Amicon or Millipore Pellicon ultrafiltration unit. Following the concentration step, the concentrate can be applied to a purification matrix such as a gel filtration medium.

Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. In addition, a chromatofocusing step can be employed. Alternatively, a hydrophobic interaction chromatography step can be employed. Suitable matrices can be phenyl or octyl moieties bound to resins. In addition, affinity chromatography with a matrix which selectively binds the recombinant protein can be employed. Examples of such resins employed are lectin columns, dye columns, antibody columns, and metal-chelating columns. Finally, one or more reversed-phase high performance liquid chromatography (RP-HPLC) steps employing hydrophobic RP-HPLC media, (e.g., silica gel or polymer resin having pendant methyl, octyl, octyldecyl or other aliphatic groups) can be employed to further purify the polypeptides. Some or all of the foregoing purification steps, in various combinations, are well known and can be employed to provide an isolated and purified recombinant protein.

It is also possible to utilize an affinity column comprising a polypeptide-binding protein of the invention, such as a monoclonal antibody generated against polypeptides of the invention, to affinity-purify expressed polypeptides. These polypeptides can be removed from an affinity column using conventional techniques, e.g., in a high salt elution buffer and then dialyzed into a lower salt buffer for use or by changing pH or other components depending on the affinity matrix utilized, or be competitively removed using the naturally occurring substrate of the affinity moiety, such as a polypeptide derived from the invention.

In this aspect of the invention, polypeptide-binding proteins, such as the anti-polypeptide antibodies of the invention or other proteins that may interact with the polypeptide of the invention, can be bound to a solid phase support such as a column chromatography matrix or a similar substrate suitable for identifying, separating, or purifying cells that express polypeptides of the invention on their surface. Adherence of polypeptide-binding proteins of the invention to a solid phase contacting surface can be accomplished by any means, for example, magnetic microspheres can be coated with these polypeptide-binding proteins and held in the incubation vessel through a magnetic field. Suspensions of cell mixtures are contacted with the solid phase that has such polypeptide-binding proteins thereon. Cells having polypeptides of the invention on their surface bind to the fixed polypeptide-binding protein and unbound cells then are washed away. This affinity-binding method is useful for purifying, screening, or separating such polypeptide-expressing cells from solution. Methods of releasing positively selected cells from the solid phase are known in the art and encompass, for example, the use of enzymes. Such enzymes are preferably non-toxic and non-injurious to the cells and are preferably directed to cleaving the cell-surface binding partner.

Alternatively, mixtures of cells suspected of containing polypeptide-expressing cells of the invention first can be incubated with a biotinylated polypeptide-binding protein of the invention. Incubation periods are typically at least one hour in duration to ensure sufficient binding to polypeptides of the invention. The resulting mixture then is passed through a column packed with avidin-coated beads, whereby the high affinity of biotin for avidin provides the binding of the polypeptide-binding cells to the beads. Use of avidin-coated beads is known in the art. See Berenson, et al. J. Cell. Biochem., 10D:239 (1986). Conventional methods are used to wash the unbound material from the column and to release bound cells from the column.

The desired degree of purity depends on the intended use of the protein. A relatively high degree of purity is desired when the polypeptide is to be administered in vivo, for example. In such a case, the polypeptides are purified so that no protein bands corresponding to other proteins are detectable upon analysis by SDS-polyacrylamide gel electrophoresis (SDS-PAGE). It will be recognized by one skilled in the pertinent field that multiple bands corresponding to the polypeptide may be visualized by SDS-PAGE, due to differential post-translational modification, processing, and the like. Most preferably, the polypeptide of the invention is purified to substantial homogeneity, as indicated by a single protein band upon analysis by SDS-PAGE. The protein band may be visualized by silver staining, Coomassie blue staining, or (if the protein is radiolabeled) by autoradiography.

Use of GNK/sGNK Nucleic Acid or Oligonucleotides

In addition to being used to express polypeptides as described above, the nucleic acids of the invention, including DNA, and oligonucleotides thereof can be used:

as probes to identify nucleic acid encoding proteins having the ability to regulate angiogenesis;

as probes to identify nucleic acid encoding protein agonists and antagonists or sGNK and related signaling pathways;

as single-stranded sense or antisense oligonucleotides, to inhibit expression of polypeptide encoded by the GNK or sGNK gene;

to further elucidate and characterize the biological activities of GNK and sGNK; and

for gene therapy.

Probes to Identify DNA Sequences Encoding Proteins Related to the Regulation of Vascularization

Among the uses of nucleic acids of the invention is the use of fragments as probes or primers. Such fragments generally comprise at least about 17 contiguous nucleotides of a DNA sequence. In other embodiments, a DNA fragment comprises at least 30, or at least 60, contiguous nucleotides of a DNA sequence.

Because homologs of SEQ ID NO: 1 or SEQ ID NO: 3, from other mammalian species, are contemplated herein, probes based on the human DNA sequence of SEQ ID NO: 1 or SEQ ID NO: 3 may be used to screen cDNA libraries derived from other mammalian species, using conventional cross-species hybridization techniques.

Using knowledge of the genetic code in combination with the amino acid sequences set forth above, sets of degenerate oligonucleotides can be prepared. Such oligonucleotides are useful as primers, e.g., in polymerase chain reactions (PCR), whereby DNA fragments are isolated and amplified.

Probes to Identify Specific Agonists and Antagonists of sGNK

The present invention also provides methods of detecting agonists and antagonists of sGNK and the GNK-sGNK complex. In one embodiment, the invention thus generally provides a method for identifying gene products that associate with sGNK comprising: (a) introducing nucleic acid sequences encoding a sGNK, or fragment thereof, into a first expression vector such that sGNK sequences are expressed as part of a fusion protein comprising a functionally incomplete first portion of a protein that is essential to the viability of a host cell; (b) introducing the nucleic acid sequences encoding a plurality of candidate gene products that interact or associate with sGNK into a second expression vector such that any candidate gene products are expressed as part of a fusion protein comprising a second functionally incomplete portion of the protein that is essential to the viability of the host cell; (c) introducing the first and second expression vectors into a host cell under suitable conditions and for a sufficient time so that host cell survival depends upon the reconstitution of both first and second functionally incomplete portions of the protein (that is essential to the viability of the host cell) into a functionally complete protein; and (d) identifying the nucleic acid sequences encoding the candidate gene products that associate with sGNK in the second expression vector.

For example, the yeast two-hybrid system (Fields and Song, Nature 340:245, 1989; U.S. Pat. No. 5,283,173) can be used to detect interactions between sGNK and other proteins or between sGNK and selected compounds, or pools of compounds, that are suspected of increasing or decreasing the activity of sGNK or of otherwise employing sGNK to transduce a biological signal. Such interactions can be detected by screening for functional reconstitution of a yeast transcription factor.

Briefly, the yeast two hybrid system was developed as a way to test whether two proteins associate or interact directly with each other and was then modified to serve as a method to “capture” candidate proteins that interact with a known protein of interest or “bait”. The bait protein is expressed as a fusion protein with the DNA-binding domain of GAL4, a yeast transcription factor, in a specially designed yeast strain (Y190) containing reporter genes under GAL4 control. (Durfee et al., Genes & Devel. 7:555, 1993). GAL 4 is a modular yeast transcription factor with the DNA binding domain confined to the N-terminal 147 residues while the transcriptional activation function resides entirely in the C-terminal 114 residues. Libraries used in the two-hybrid system have clones expressing GAL4 activation domain fusion proteins. The method detects the reconstitution of GALA function when two fusion proteins encode proteins that associate with each other, so that the DNA-binding domain fusion recruits the activation domain fusion into position at the GALA promoter, leading to transcriptional activation of the GAL4-controlled reporter genes.

The sGNK nucleic acid sequences disclosed herein can be cloned into a suitable vector carrying the DNA-binding domain of GALA and transformed into an appropriate yeast strain to produce yeast cells which express a GAL4 DNA-binding domain/sGNK region fusion protein using methods well known in the art. Activation domain cDNA libraries can then be screened in appropriate vectors. A positive signal in such a two-hybrid assay can result from cDNA clones that encode proteins that specifically associate with sGNK, such as substrates or activators of sGNK. Knowledge of proteins that associate with sGNK can also permit searching for inhibitors of downstream signaling pathways.

The functional interaction between sGNK and its associating proteins also permits screening for small molecules that interfere with the GNK/sGNK, sGNK/substrate, or sGNK/activator association and thereby inhibit signal transduction via the GNK-sGNK pathway. For example, the yeast two-hybrid system can be used to screen for signaling pathway inhibitors as follows.

sGNK and its activator/inhibitor, or portions thereof responsible for their interaction, can be fused to the GALA DNA binding domain and GAL4 transcriptional activation domain, respectively, and introduced into a strain that depends on GAL4 activation for growth on plates lacking histidine. Compounds that prevent growth can be screened in order to identify inhibitors of the GNK-sGNK pathway or sGNK biological activity. Alternatively, the screen can be modified so that sGNK/activator or sGNK/substrate interaction inhibits growth, so that inhibition of the interaction allows growth to occur. Another approach to in vitro screening for inhibition of sGNK biological activity would be to immobilize one of the components, such as sGNK, or portions thereof, in wells of a microtiter plate, and to couple an easily detected indicator to the other component. An inhibitor of the interaction is identified by the absence of the detectable indicator in the well.

A high throughput screening assay can also be utilized to identify compounds that inhibit sGNK activity. For example, natural product extracts, from plant and marine sources, as well as microbial fermentation broths, can be sources of kinase inhibitors and can be screened for potential sGNK antagonists. Other sources of sGNK antagonists include pre-existing or newly generated libraries of small organic molecules and pre-existing or newly generated combinatorial chemistry libraries. Identification of endogenous sGNK substrate(s), and mapping of their interactive site(s) to determine their specific recognition motif(s), can enable the development of peptide mimetic inhibitors. In addition, in vivo regulation of sGNK activity likely involves endogenous protein inhibitor(s), which can be identified using the assay(s) described herein.

These assays also facilitate the identification of other molecules that interact with sGNK in a physiologically relevant manner, such as endogenous substrates, activators and the aforementioned natural protein inhibitors. Such molecules include, but are not limited to, receptors and receptor associated polypeptides, guanine nucleotide binding proteins (G proteins), GEFs, guanine nucleotide activating proteins (GAPs), transcription activators, and repressors. Additionally, the sGNK assays can serve as readouts to identify other enzymes involved in the signaling cascade, such as other kinases, phosphatases, and phospholipases.

Sense-antisense

Other useful fragments of the nucleic acids include antisense or sense oligonucleotides comprising a single-stranded nucleic acid sequence (either RNA or DNA) capable of binding to target mRNA (sense) or DNA (antisense) sequences. Antisense or sense oligonucleotides, according to the present invention, comprise a fragment of SEQ ID NO: 1 or SEQ ID NO: 3. Such a fragment generally comprises at least about 14 nucleotides, preferably from about 14 to about 30 nucleotides. The ability to derive an antisense or a sense oligonucleotide, based upon a cDNA sequence encoding a given protein is described in, for example, Stein and Cohen (Cancer Res. 48:2659, 1988) and van der Krol et al. (Bio/Techniques 6:958, 1988).

Binding of antisense or sense oligonucleotides to target nucleic acid sequences results in the formation of duplexes that block or inhibit protein expression by one of several means, including enhanced degradation of the mRNA by RNAse H, inhibition of splicing, premature termination of transcription or translation, or by other means. Antisense or sense oligonucleotides further comprise oligonucleotides having modified sugar-phosphodiester backbones (or other sugar linkages, such as those described in WO91/06629) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences.

Other examples of sense or antisense oligonucleotides include those oligonucleotides which are covalently linked to organic moieties, such as those described in WO 90/10448, and other moieties that increases affinity of the oligonucleotide for a target nucleic acid sequence, such as poly-(L-lysine). Further still, intercalating agents, such as ellipticine, and alkylating agents or metal complexes may be attached to sense or antisense oligonucleotides to modify binding specificities of the antisense or sense oligonucleotide for the target nucleotide sequence.

Antisense or sense oligonucleotides may be introduced into a cell containing the target nucleic acid sequence by any gene transfer method, including, for example, lipofection, CaPO₄-mediated DNA transfection, electroporation, or by using gene transfer vectors such as Epstein-Barr virus.

Sense or antisense oligonucleotides also may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell.

Alternatively, a sense or an antisense oligonucleotide may be introduced into a cell containing the target nucleic acid sequence by formation of an oligonucleotide-lipid complex, as described in WO 90/10448. The sense or antisense oligonucleotide-lipid complex is preferably dissociated within the cell by an endogenous lipase.

Further Elucidation and Characterization of the Biological Activities of GNK and sGNK

The materials and methods of the present invention may be used to prepare cells, embryos, fetuses, and animals that are heterozygous (+/−) or homozygous (−/−) for a GNK or sGNK targeted mutation. These cells, embryos, fetuses, and animals are useful for demonstrating the role of GNK and/or sGNK in vascularization and for demonstrating other biological activities of GNK and/or sGNK. The skilled artisan will realize that a variety of methods may be used to generate cells, embryos, fetuses, and animals with alterations in the expression of GNK and/or sGNK. These methods include generating targeted mutations and knockouts, attenuating gene expression using antisense, ribozyme, or small molecule technology, and attenuating or activating gene expression using a Zn finger approach such as that described by Segal et al. (Proc. Nat. Acad. Sci. USA 96(6):2758, 1999). For the latter approach, Zn fingers are targeted 5′ or 3′ of the coding portion of GNK or sGNK, or within an intron, allowing the novel construction of a novel gene switch.

Gene Therapy

The invention also provides expression vectors useful in gene therapy applications. Appropriate expression vectors are readily constructed by those skilled in the art and may be used for gene therapy using retroviral vector constructs or may be developed and utilized with other viral constructs including, for example, poliovirus (Evans et al., Nature 339:385, 1989; Sabin, J. Biol. Standard. 1:115, 1973); rhinovirus; poxviruses, such as canary pox or vaccinia virus (Fisher-Hoch et al., Proc. Natl. Acad. Sci. USA 86:317, 1989; Flexner et al., Ann. N.Y. Acad. Sci. 569:86, 1989; Flexner et al., Vaccine 8:17, 1990; U.S. Pat. Nos. 4,603,112 and 4,769,330; WO 89/01973); polyoma viruses such as SV40 (Mulligan et al., Nature 277:108, 1979); influenza virus (Luytjes et al., Cell 59:1107, 1989; McMichael et al., N. Eng. J. Med. 309:13, 1983: Yap et al., Nature 273:238, 1978); adenoviruses (Berkner, Biotechniques 6:616, 1988; Rosenfeld et al., Science 252:431, 1991); parvoviruses such as adeno-associated virus (Samulski et al., J. Virol. 63:3822, 1989; Mendelson et al., Virol. 166:154, 1988) and herpes viruses (Kit, Adv. Exp. Med. Biol. 215:219, 1989).

Once a vector has been prepared, it may be therapeutically administered by well known methods, for example, by direct administration, or via transfection utilizing physical methods, such as lipofection (Felgner et al., Proc. Natl. Acad. Sci. USA 84:7413, 1989), direct DNA injection (Ascadi et al., Nature 352:815, 1991), microprojectile bombardment (Williams et al., Proc. Natl. Acad. Sci. USA 88:2726, 1991), liposomes (Wang et al., Proc. Natl. Acad. Sci. USA 84:7851, 1987), calcium phosphate (Dubensky et al., Proc. Natl. Acad. Sci. USA 81:7529, 1984), or DNA ligand (Wu et al., J. Biol. Chem. 264:16985, 1989).

Use of GNK and/or sGNK Polypeptides and Fragmented Polypeptides

Uses include, but are not limited to, the following:

Purification Reagents

Measuring Biological Activity

Identification of Agonists or Antagonists of GNK or sGNK

Identification of Unknown Proteins

Antibodies

Therapeutic Agents

Purification Reagents

The GNK or sGNK polypeptides of the invention find use as protein purification reagents. For example the GNK polypeptides may be attached to a solid support material and used to purify sGNK proteins by affinity chromatography, or vice versa, i.e., the sGNK is attached to a solid support and used to purify GNK. In particular embodiments, a sGNK polypeptide (in any form described herein that is capable of binding GNK), or vice versa, is attached to a solid support by conventional procedures. As one example, chromatography columns containing functional groups that will react with functional groups on amino acid side chains of proteins are available (Pharmacia Biotech, Inc., Piscataway, N.J.). In an alternative, a polypeptide/Fc protein (as discussed above) is attached to Protein A- or Protein G-containing chromatography columns through interaction with the Fc moiety.

In addition to purification, such affinity columns can be used to select and isolate previously unidentified binding proteins, moieties, and/or cofactors.

Measuring Biological Activity

GNK polypeptides also find use in measuring the biological activity of sGNK protein in terms of their binding affinity, and vice versa. The polypeptides thus may be employed by those conducting “quality assurance” studies, e.g., to monitor shelf life and stability of protein under different conditions. For example, the GNK polypeptides may be employed in a binding affinity study to measure the biological activity of sGNK that has been stored at different temperatures, or produced in different cell types, or vice versa. The sGNK may also be used to determine whether biological activity is retained after modification of GNK (e.g., chemical modification, truncation, mutation, etc.), and vice versa. The binding affinity of the modified GNK/sGNK protein is compared to that of an unmodified GNK/sGNK protein to detect any adverse impact of the modifications on biological activity of GNK/sGNK. The biological activity of a GNK/sGNK protein thus can be ascertained before it is used in a research study of angiogenesis, for example.

Identification of Agonists and Antagonists of GNK or sGNK

The polypeptides of the present invention may also be used in a screening assay to identify compounds and small molecules which inhibit (antagonize) or enhance (agonize) activation of the polypeptides of the instant invention. Thus, for example, polypeptides of the invention may be used to identify antagonists and agonists from cells, cell-free preparations, chemical libraries, and natural product mixtures. The antagonists and agonists may be natural or modified substrates, ligands, enzymes, receptors, etc. of the polypeptides of the instant invention, or may be structural or functional mimetics of the polypeptides. Potential antagonists of the polypeptides of the instant invention may include small molecules, peptides, and antibodies that bind to and occupy a binding site of the polypeptides, causing them to be unavailable to bind to their ligands and therefore preventing normal biological activity. Other potential antagonists are antisense molecules which may hybridize to mRNA in vivo and block translation of the mRNA into the polypeptides of the instant invention. Potential agonists include small molecules, peptides and antibodies which bind to the instant polypeptides and elicit the same or enhanced biological effects as those caused by the binding of the polypeptides of the instant invention.

Small molecule agonists and antagonists are usually less than 10K molecular weight and may possess a number of physiochemical and pharmacological properties that enhance cell penetration, resist degradation and prolong their physiological half-lives. (Gibbs, J., Pharmaceutical Research in Molecular Oncology, Cell, Vol. 79 (1994).) Antibodies, which include intact molecules as well as fragments such as Fab and F(ab′)2 fragments, may be used to bind to and inhibit the polypeptides of the instant invention by blocking the commencement of a signaling cascade. It is preferable that the antibodies are humanized, and more preferable that the antibodies are human. The antibodies of the present invention may be prepared by any of a variety of well-known methods.

Specific screening methods are known in the art and along with integrated robotic systems and collections of chemical compounds/natural products are extensively incorporated in high throughput screening so that large numbers of test compounds can be tested for antagonist or agonist activity within a short amount of time. These methods include homogeneous assay formats such as fluorescence resonance energy transfer, time resolved fluorescence resonance energy transfer, fluorescence polarization, scintillation proximity assays, reporter gene assays, fluorescence quenched enzyme substrate, chromogenic enzyme substrate and electrochemiluminesence, as well as, more traditional heterogeneous assay formats such as enzyme linked immunosorbent assays (ELISA) or radioimmunoassays. Homogeneous assays are mix and read style assays that are very amenable to robotic application, whereas heterogeneous assays require separation of free from bound analyte by more complex unit operations such as filtration, centrifugation or washing. These assays are utilized to detect a wide variety of specific biomolecular interactions and the inhibition thereof by small organic molecules, including protein-protein, receptor-ligand, enzyme-substrate, etc. These assay methods and techniques are well known in the art (see, e.g., High Throughput Screening: The Discovery of Bioactive Substances, John P. Devlin (ed.), Marcel Dekker, New York, 1997, ISBN: 0-8247-0067-8; http://www.lab-robotics.org/; http://www.sbsonline.org/). The screening assays of the present invention are amenable to high throughput screening of chemical libraries and are suitable for the identification of small molecule drug candidates, antibodies, peptides and other antagonists and/or agonists.

One embodiment of a method for identifying molecules which antagonize or inhibit the polypeptides involves adding a candidate molecule to a medium which contains cells that express the polypeptides of the instant invention; changing the conditions of said medium so that, but for the presence of the candidate molecule, the polypeptides would be bound to their ligands; and observing the binding and stimulation or inhibition of a functional response. The activity of the cells which were contacted with the candidate molecule may then be compared with the identical cells which were not contacted and agonists and antagonists of the polypeptides of the instant invention may be identified. The measurement of biological activity may be performed by a number of well-known methods such as measuring the amount of protein present (e.g. an ELISA) or of the protein's activity. A decrease in biological stimulation or activation would indicate an antagonist. An increase would indicate an agonist.

Screening assays can further be designed to find molecules that mimic the biological activity of the polypeptides of the instant invention. Molecules which mimic the biological activity of a polypeptide may be useful for enhancing the biological activity of the polypeptide. To identify compounds for therapeutically active agents that mimic the biological activity of a polypeptide, it must first be determined whether a candidate molecule binds to the polypeptide. A binding candidate molecule is then added to a biological assay to determine its biological effects. The biological effects of the candidate molecule are then compared to the those of the polypeptide.

Identification of Unknown Proteins

As set forth above, a polypeptide or peptide fingerprint can be entered into or compared to a database of known proteins to assist in the identification of the unknown protein using mass spectrometry (W. J. Henzel et al., Proc. Natl. Acad. Sci. USA 90:5011-5015, 1993; D. Fenyo et al., Electrophoresis 19:998-1005, 1998). A variety of computer software programs to facilitate these comparisons are accessible via the Internet, such as Protein Prospector (Internet site: prospector.uscf.edu), MultiIdent (Internet site: www.expasy.ch/sprot/multiident.html), PeptideSearch (Internet site: www.mann.embl-heiedelberg.de . . . deSearch/FR_PeptideSearch Form.html), and ProFound (Internet site: www.chait-sgi.rockefeller.edu/cgi-bin/prot-id-frag.html). These programs allow the user to specify the cleavage agent and the molecular weights of the fragmented peptides within a designated tolerance. The programs compare these molecular weights to protein databases to assist in determining the identity of the unknown protein.

In addition, a polypeptide or peptide digest can be sequenced using tandem mass spectrometry (MS/MS) and the resulting sequence searched against databases (J. K. Eng, et al., J. Am. Soc. Mass Spec. 5:976-989 (1994); M. Mann and M. Wilm, Anal. Chem. 66:4390-4399 (1994); J. A. Taylor and R. S. Johnson, Rapid Comm. Mass Spec. 11:1067-1075 (1997)). Searching programs that can be used in this process exist on the Internet, such as Lutefisk 97 (Internet site: www.lsbc.com:70/Lutefisk97.html), and the Protein Prospector, Peptide Search and ProFound programs described above. Therefore, adding the sequence of a gene and its predicted protein sequence and peptide fragments to a sequence database can aid in the identification of unknown proteins using tandem mass spectrometry.

Antibodies

Antibodies that are immunoreactive with the polypeptides of the invention are provided herein. Such antibodies specifically bind to the polypeptides via the antigen-binding sites of the antibody (as opposed to non-specific binding). Thus, the polypeptides, fragments, variants, fusion proteins, etc., as set forth above may be employed as “immunogens” in producing antibodies immunoreactive therewith. More specifically, the polypeptides, fragment, variants, fusion proteins, etc. contain antigenic determinants or epitopes that elicit the formation of antibodies.

These antigenic determinants or epitopes can be either linear or conformational (discontinuous). Linear epitopes are composed of a single section of amino acids of the polypeptide, while conformational or discontinuous epitopes are composed of amino acid sections from different regions of the polypeptide chain that are brought into close proximity upon protein folding (C. A. Janeway, Jr. and P. Travers, Immuno Biology 3:9 (Garland Publishing Inc., 2nd ed. 1996)). Because folded proteins have complex surfaces, the number of epitopes available is quite numerous; however, due to the conformation of the protein and steric hinderances, the number of antibodies that actually bind to the epitopes is less than the number of available epitopes (C. A. Janeway, Jr. and P. Travers, Immuno Biology 2:14 (Garland Publishing Inc., 2nd ed. 1996)). Epitopes may be identified by any of the methods known in the art.

Thus, one aspect of the present invention relates to the antigenic epitopes of the polypeptides of the invention. Such epitopes are useful for raising antibodies, in particular monoclonal antibodies, as described in more detail below. Additionally, epitopes from the polypeptides of the invention can be used as research reagents, in assays, and to purify specific binding antibodies from substances such as polyclonal sera or supernatants from cultured hybridomas. Such epitopes or variants thereof can be produced using techniques well known in the art such as solid-phase synthesis, chemical or enzymatic cleavage of a polypeptide, or using recombinant DNA technology.

As to the antibodies that can be elicited by the epitopes of the polypeptides of the invention, whether the epitopes have been isolated or remain part of the polypeptides, both polyclonal and monoclonal antibodies may be prepared by conventional techniques. See, for example, Monoclonal Antibodies, Hybridomas: A New Dimension in Biological Analyses, Kennet et al. (eds.), Plenum Press, New York (1980); and Antibodies: A Laboratory Manual, Harlow and Land (eds.), Cold Spring Harbor Laboratory Press, Cold Spring Harbor, N.Y., (1988).

Hybridoma cell lines that produce monoclonal antibodies specific for the polypeptides of the invention are also contemplated herein. Such hybridomas may be produced and identified by conventional techniques. One method for producing such a hybridoma cell line comprises immunizing an animal with a polypeptide; harvesting spleen cells from the immunized animal; fusing said spleen cells to a myeloma cell line, thereby generating hybridoma cells; and identifying a hybridoma cell line that produces a monoclonal antibody that binds the polypeptide. The monoclonal antibodies may be recovered by conventional techniques.

The monoclonal antibodies of the present invention include chimeric antibodies, e.g., humanized versions of murine monoclonal antibodies. Such humanized antibodies may be prepared by known techniques and offer the advantage of reduced immunogenicity when the antibodies are administered to humans. In one embodiment, a humanized monoclonal antibody comprises the variable region of a murine antibody (or just the antigen binding site thereof) and a constant region derived from a human antibody. Alternatively, a humanized antibody fragment may comprise the antigen binding site of a murine monoclonal antibody and a variable region fragment (lacking the antigen-binding site) derived from a human antibody. Procedures for the production of chimeric and further engineered monoclonal antibodies include those described in Riechmann et al. (Nature 332:323, 1988), Liu et al. (PNAS 84:3439, 1987), Larrick et al. (Bio/Technology 7:934, 1989), and Winter and Harris (TIPS 14:139, May, 1993).

Procedures that have been developed for generating human antibodies in non-human animals may be employed in producing antibodies of the present invention. The antibodies may be partially human or preferably completely human. For example, transgenic mice into which genetic material encoding one or more human immunoglobulin chains has been introduced may be employed. Such mice may be genetically altered in a variety of ways. The genetic manipulation may result in human immunoglobulin polypeptide chains replacing endogenous immunoglobulin chains in at least some, and preferably virtually all, antibodies produced by the animal upon immunization.

Mice in which one or more endogenous immunoglobulin genes have been inactivated by various means have been prepared. Human immunoglobulin genes have been introduced into the mice to replace the inactivated mouse genes. Antibodies produced in the animals incorporate human immunoglobulin polypeptide chains encoded by the human genetic material introduced into the animal.

Examples of techniques for the production and use of such transgenic animals are described in U.S. Pat. Nos. 5,814,318, 5,569,825, and 5,545,806, which are incorporated by reference herein.

Antigen-binding fragments of the antibodies, which may be produced by conventional techniques, are also encompassed by the present invention. Examples of such fragments include, but are not limited to, Fab and F(ab′)₂ fragments. Antibody fragments and derivatives produced by genetic engineering techniques are also provided.

In one embodiment, the antibodies are specific for the polypeptides of the present invention and do not cross-react with other proteins. Screening procedures by which such antibodies may be identified are well known, and may involve immunoaffinity chromatography, for example.

Therapeutic Agents

When used as a therapeutic agent, sGNK, a sGNK antagonist, or a sGNK agonist can be formulated into pharmaceutical compositions according to known methods, either individually, in combination, or combined with GNK, an GNK agonist, or an GNK agonist (either individually or in combinations). The sGNK, its antagonist, or agonist can be introduced into the intracellular environment using methods well known in the field, such as encasing sGNK in liposomes or coupling sGNK to a monoclonal antibody targeted to a specific cell type.

The sGNK, a sGNK antagonist, or a sGNK agonist can be combined in admixture, either as the sole active material or with other known active materials, with pharmaceutically suitable diluents (e.g., Tris-HCl, acetate, or phosphate buffers), preservatives (e.g., Thimerosol, benzyl alcohol, parabens), emulsifiers, solubilizers, adjuvants and/or carriers. Suitable carriers and their formulations are described in Remington's Pharmaceutical Sciences, 16^{th} ed. 1980, Mack Publishing Co. In addition, such compositions can contain sGNK, its antagonist, or its agonist, complexed with polyethylene glycol (PEG), metal ions, or incorporated into polymeric compounds such as polyacetic acid, polyglycolic acid, hydrogels, etc., or incorporated into liposomes, microemulsions, micelles, unilamellar or multilamellar vesicles, erythrocyte ghosts or spheroplasts. Such combinations will influence the physical state, solubility, stability, rate of in vivo release, and rate of in vivo clearance,

The following examples are provided to further illustrate particular embodiments of the invention, and are not to be construed as limiting the scope of the present invention.

### EXAMPLE 1

### Purification of sGNK

This example describes purification of sGNK from rabbit lungs. Lungs were isolated from seventy New Zealand white rabbits intravenously injected with 100 μg/kg of human recombinant IL-1α, fifteen minutes prior to sacrifice. Following sacrifice, lungs were rapidly removed, washed in conventional ice cold phosphate buffered saline (cold PBS), immediately fast frozen, and stored at −80° C. The lungs were homogenized using a Brinkman tissue homogenizer. Tissue and cellular debris was removed by centrifugation and ultrafiltration. The resulting supernatant was made 25% with respect to ammonium sulphate and proteins precipitated by this 0-25% salt cut were collected by centrifugation. Pelleted proteins were resuspended and sequentially subjected to the following purification steps: (1) ion-exchange chromatography using Source 15 Q (Pharmacia); (2) dye affinity chromatography using Reactive Green 19 (Sigma Chemicals); (3) size exclusion chromatography using Superdex 200 (Pharmacia); (4) affinity chromatography using heparin-sepharose (Pharmacia); (5) ion-exchange chromatography using Mono Q resin (Pharmacia); (6) size exclusion chromatography using SEC-400 (BioRad); (7) ion-exchange chromatography using a microbore Mono Q column; and electrophoretic separation using SDS-PAGE with 8-16% polyacrylamide gradient gels (Novex).

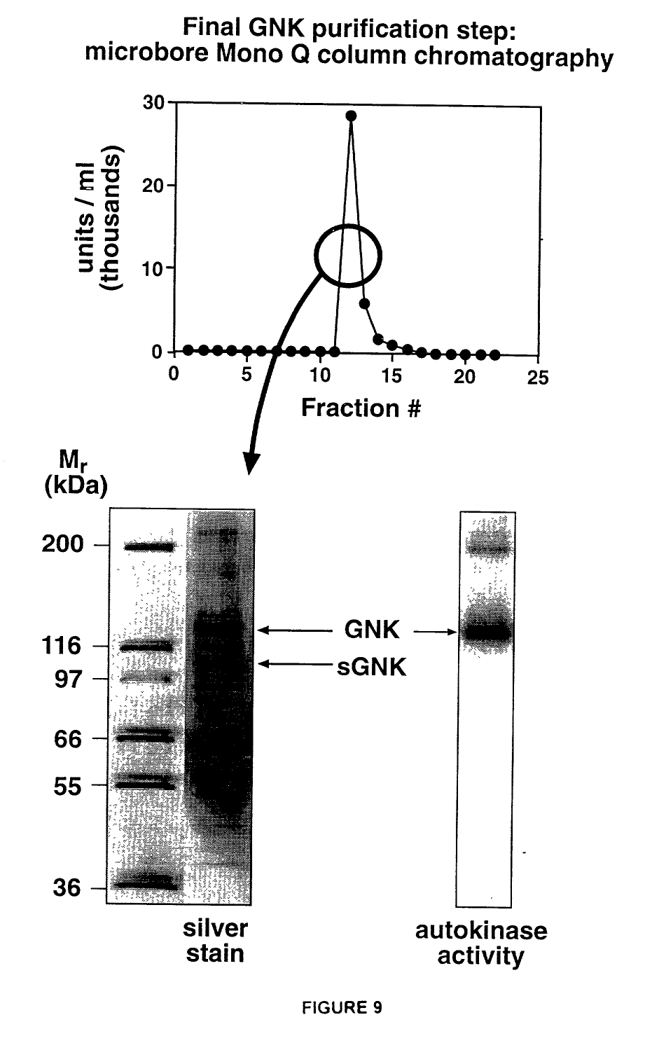

The final chromatographic step, fractionation on a microbore MonoQ column containing 35 μl resin, was performed to concentrate the sample in a small volume for electrophoresis (FIG. 9). Briefly, fractions containing both GNK and sGNK from the SEC-400 chromatography step, were loaded onto the Mono Q column, previously equilibrated in 20 mM Tris-HCl, pH 8.5, 10 mM β-glycerophosphate, 1 mM dithiothreitol (DTT), 1 mM EDTA, 1 mM EGTA, 1 mM phenylmethyl sulphonyl fluoride (PMSF), 0.1 mM leupeptin, 10% glycerol and 0.1% NP-40 (Buffer A), at a flow rate of 50 μl/min. After loading, the column was washed with 10 column volumes of Buffer A. Bound proteins, which included GNK and sGNK, were eluted using a 500 μl linear 0-500 mM NaCl gradient in Buffer A.

The fraction containing GNK and sGNK was subjected to SDS-PAGE on an 8-16% Tris-glycine gradient gel (Novex). Bands were visualized by silver staining. sGNK was identified and excised from the gel. Trypsin digestion was performed in situ and resulting peptides were extracted by methods known in the art. The isolated peptides were analyzed by mass spectroscopy. The amino acid sequences of several peptides were ascertained and these sequences were utilized to design oligonucleotide probes for use in the molecular cloning of sGNK.

### EXAMPLE 2

### Cloning and Sequencing of Human sGNK

The amino acid sequence for sGNK from the rabbit HBCK preparation was obtained using protease digestion and mass spectrometry, using methods known in the art. Three rabbit peptide sequences were found that matched a publicly-available human genomic expressed sequence containing 71 amino acids (Genbank accession number T11454). Two oligonucleotide primers were used to screen cDNA libraries to determine whether they contained the message encoding the 71 amino acid sequence. These primers, identified as primer 21497 and 21499, were: 5′-

| | |
|---|---|
| 5′-GCCTTTGGACAAGCACACAC-3′ | (SEQ ID NO: 11) and |
| 5′-CTCCTTCAGCTCCTGGGCC-3′ | (SEQ ID NO: 12), |

respectively. Several human cDNA libraries were found to be positive, including Raji (B cell), Clone 22 (T cell), KB (epithelial cell), NK cell (NK cell), human dermal fibroblasts (HDF), and W126 (lung fibroblast).

To generate a template for a probe, the Xgt 10 KB library was amplified using primers 21497 and 21499. Amplifying 7.5 nanograms (ng) of the template with primer 21499 resulted in an 200 base pair single-stranded antisense PCR probe. This probe was used to screen 400,000 plaques from a human KB library made in λgt10. Four positive plaques were identified.

One of the positive plaques, clone KB-9-2-1, was mapped by PCR using all combinations of one vector and one insert primer. The vector primer sequences, referred to as U30 and D30, were:

| | |
|---|---|
| 5′-CGAGCTGCTCTATAGACTGCTGGGTAGTCC-3′ | (SEQ ID NO: 13) and |
| 5′-TAACAGAGGTGGCTTATGAGTATTTCTTCC-3′ | (SEQ ID NO: 14), |

respectively. Primers 21497 and 21499 were used as insert primers.

Sequencing of the PCR products revealed that product 2-2 (produced using primers U30 and 21499) extended the sequence in the 5′ direction and that product 2-5 (produced using primers D30 and 21497) extended the sequence in the 3′ direction. A probe was made from PCR product 2-5 by digesting with EcoRI and amplification with primer 21498, 5′-AAACCACAAGAAGGTGGCTG-3′ (SEQ ID NO: 15). This probe was used to screen 500,000 plaques from a Raji cDNA library in λgt10. Four positive clones were picked and sequenced using conventional sequencing procedures. A Raji probe from one of these clones (Raji9-9-1A) was generated by amplifying with primers 23206 and 23207,

| | |
|---|---|
| 5′-AGGTGAAGCGGCTGTCCCACGA-3′ | (SEQ ID NO: 16) and |
| 5′-CTCCTTCAGCTCCTGGGCCACA-3′ | (SEQ ID NO: 17), |

respectively. Amplifying 24 ng of clone Raji9-9-1A using primer 23207 generated an antisense 35 bp single-stranded probe. This probe was used to screen 500,000 plaques from a HDF library made in λgt10. Three positive clones were identified and sequenced.

The entire sGNK open reading frame is present in a composite of the four Raji clones and the three HDF clones. Further, Raji9-9-1A was found to be a full length clone. This clone was used as the template to make expression constructs.

### EXAMPLE 3

### Kinase Assay Demonstrating Autophosphorylation of GNK and Phoshorylation of sGNK by GNK

GNK autophosphorylation was demonstrated for both purified rabbit and human recombinant GNK by incubating GNK in the presence of γ-³²P-ATP and Mn²⁺. Kinase assays were performed in kinase buffer (20 mM HEPES, pH 7.4, 10 mM MnCl₂ 25 μM cold ATP and 1 μCi gamma-³²P-ATP), at 30° C. for 20 minutes. The reactions were stopped by adding conventional SDS electrophoresis buffer followed by incubation of the mixture at 100° C. for 3 minutes. Reaction products were separated by electrophoresis on 8-16% gradient gels. These gels were silver stained, dried, then exposed to storage phosphor screens which were analyzed using a Molecular Dynamics Phosphoimager.

GNK-mediated phosphorylation of sGNK was demonstrated using the kinase assay described above, except purified, recombinant sGNK was added to the kinase reaction mixture. Electrophoretic and Phosphoimager analysis of the reaction products were performed as described

### EXAMPLE 4

### Isolation of a Genomic Clone Encoding a Portion of the Murine GNK Gene and Construction of an GNK Gene Targeting Vector

A lambda genomic library prepared from 129 DNA (Stratagene, La Jolla, Calif.) was screened with a human GNK cDNA. The insert from a hybridizing phage was subcloned as a Not I restriction fragment into pGEM 11 and mapped by a combination of sequencing, restriction mapping, and PCR analyses using primers based on the human GNK cDNA sequence. A homologous recombination vector was constructed in which an EcoRI fragment containing what we assume to be exon 1 was replaced with a PGK-neo cassette. The 5′ end of the targeting vector extends to an Asp718 site 5′ of exon 1 and the 3′ end of the targeting vector extends to an EcoR1 site 3′ of what we assume to be exon 2. Additionally, an MC-TK cassette was subcloned into the 3′ end of the vector. The backbone of the targeting vector is pGEM 11. The PGK-neo and MC-TK cassettes are standard cassettes that confer, respectively, resistance to G418 and sensitivity to ganciclovir (see FIG. 10).

### EXAMPLE 5

### Generation of Embryonic Stem (ES) Cell Clones Heterozygous for a Targeted Mutation in GNK

The GNK homologous recombination vector was electroporated into 129 derived embryonic stem (ES) cells maintained on irradiated primary embryonic fibroblast feeder layers in LIF containing media using standard techniques. Transfected cells were selected for 9-14 days in media containing 175 μg/ml G418 and 2 μM ganciclovir using standard techniques. Resistant clones were expanded and analyzed for the presence of a targeted GNK allele by PCR using the primers

| | |
|---|---|
| 5′-CCGGTGGATGTGGAATGTGTG-3′ | (SEQ ID NO: 5) and |
| 5′-CAAAGCCAAGGTTCATTCGGTG-3′ | (SEQ ID NO: 6) |

using equimolar concentrations of each primer. Colonies yielding the 1.4 kb PCR product expected for a targeted mutation in the GNK gene (and confirmed using the positive control vector; see FIG. 10) were expanded and used to generate chimeric mice. Additionally, genomic southern analyses using BamHI digested genomic DNA and a 1.4 kb Asp718-Not I probe isolated from the positive control vector were used to confirm targeted disruption of the GNK gene; the wild type allele yields a 8.5 kb BamHI hybridizing band and the disrupted allele yields a 6 kb band.

### EXAMPLE 6

### Generation of Chimeric Mice Using ES Cells Heterozygous for a Targeted Mutation in GNK

ES cell clones heterozygous for a targeted mutation in GNK were used to generate chimeras by blastocyst injection of day 3.5 C57BL/6 blastocysts, followed by transfer of injected blastocysts into day 2.5 pseudopregnant Swiss-Webster recipients using standard techniques. The resulting male chimeras were bred to C57BL/6 females and germline transmission events were determined by a combination of coat color analyses and PCR analyses of ear punch biopsies using standard techniques. The primers used for these four primer PCR analyses (equimolar concentration of each primer) are:

| | |
|---|---|
| 5′-GCCCTGAATGAACTGCAGGACG-3′ | (SEQ ID NO: 7) |
| 5′-CACGGGTAGCCAACGCTATGTC-3′ | (SEQ ID NO: 8) |
| 5′-CTTCCGCTTCCACGACACTCG-3′ | (SEQ ID NO: 9) |
| 5′-CTCAATGGCCTCAGACGCCAG-3′ | (SEQ ID NO: 10) |

The wild type GNK allele yields a 170 bp PCR product and the mutant GNK allele yields a 520 bp PCR product. Mice heterozygous for the targeted mutation in GNK yield both PCR products.

### EXAMPLE 7

### Generation of GNK-deficient Fetuses and Fibroblasts

Mice heterozygous for the targeted mutation in GNK (GNK+/−) were intercrossed. Fetuses were obtained at various developmental stages and genotyped using the four primers described in Example 6. Fetuses homozygous for the GNK mutation (GNK−/−) yielded only the 520 bp PCR product. Fibroblasts homozygous for the GNK mutation were derived from embryonic day 11.5-13.5 (e11.5-e13.5) fetuses obtained from GNK+/− intercrosses, genotyped as described above and cultured using standard techniques.

### EXAMPLE 8

### Oligomeriztion of GNK

Human recombinant GNK (hu-rGNK), permitted to autophosphorylate as described above, for various times (t=0, 5, 15, 30, 45, 60, 90, 120, 150, or 180 minutes) at 30° C., then analyzed by SDS-PAGE, revealed time dependent formation of high molecular weight aggregates of GNK. Based on co-electrophoresed molecular weight standards, a single silver-stained, radioactive, aggregate band migrated in the gel to a distance consistent with a moiety of ˜350 kDa, suggesting a trimeric GNK complex. Higher molecular weight aggregates were also detected.

Purified human recombinant GNK also behaves like a molecule of ˜300-350 kDa on size exclusion chromatography (gel filtration) when compared to calibration standards run under identical conditions. Briefly, hu-rGNK loaded onto a Superdex 200 column previously equilibrated with 20 mM HEPES, pH 7.4, and run at a flow rate of 2.0 ml/min elutes from the column with an apparent molecular weight of 300-350 kDa, again consistent with a trimeric complex. When the same Superdex 200 analysis was performed on autophosphorylated hu-rGNK, GNK eluted from the column as higher order oligomers (>500 kD) as well as at 300-350 kDa.

### EXAMPLE 9

### Monoclonal Antibodies to sGNK

This example illustrates the preparation of anti-sGNK monoclonal antibodies. sGNK is expressed in mammalian host cells such as COS-7 or 129 cells and purified by techniques generally known in the art. Purified or partially purified sGNK can be used to generate monoclonal antibodies against sGNK using conventional techniques, such as those described in U.S. Pat. No. 4,411,993.

Briefly, BALB/c mice are immunized with sGNK emulsified in complete Freund's adjuvant and injected subcutaneously or intraperitoneally in amounts ranging from 10-100 μg. Ten or twelve days later, the immunized animals are boosted with additional sGNK emulsified in incomplete Freund's adjuvant. Mice are boosted thereafter on a weekly to biweekly immunization schedule. Serum samples are periodically taken by retro-orbital plexus bleeding or tail tip excision for testing for sGNK antibodies using conventional dot blot assay or ELISA.

Following detection of an appropriate antibody titer, positive animals are provided one last intravenous injection of sGNK in saline. Three to four days later, the animals are sacrificed, and spleen cells are harvested and fused to a murine myeloma cell line, e.g., NS1 or preferably P3x63Ag8.653 (ATCC CRL 1580). Fusions generate hybridoma cells, which are plated in multiple microtiter in a HAT (hypoxanthine, aminopterin, thymidine) selective medium to inhibit proliferation of non-fused cells, myeloma hybrids, and spleen cell hybrids.

The hybridoma cells are screened by ELISA for reactivity against purified sGNK by adaptations disclosed in Engvall (Immunochem. 8:871, 1971) and in U.S. Pat. No. 4,700,004. A preferred screening technique is the antibody capture technique. (Beckmann et al., J. Immunol. 144:4212, 1990). Positive hybridoma cells can be injected intraperitoneally into syngeneic BALB/c mice to produce ascites fluids containing high concentrations of anti-sGNK monoclonal antibodies. Alternatively, hybridoma cells can be grown in vitro in flasks or roller bottles by various techniques. Monoclonal antibodies produced in mouse ascites fluids can be purified by ammonium sulfate precipitation, followed by gel exclusion chromatography. Alternatively, affinity chromatography based on binding of antibody to protein A or protein G can be used, as can affinity chromatography based on binding to sGNK.

### EXAMPLE 10

### Identifying Novel Genes and/or Gene Products Involved in Regulating Vascularization

Novel genes and gene products involved in regulating vascularization will be identified by comparing the gene expression or protein modification patterns in wild-type cells, organs, or animals (“wt cells”), i.e., those expressing GNK and sGNK, with GNK- and/or sGNK-deficient cells , organs, or animals (“deficient cells”).

For example, ES cells (GNK +/+) and ES null cells (GNK −/−) will be propagated in vitro in parallel cultures, i.e., under identical culture conditions. After a predetermined period of time, the cells will be harvested by scraping, trypsinization, or other methods known in the art. The harvested cells will be pelleted by low speed centrifugation and the cell culture media decanted.

The harvested cell pellets can be washed, if desired, to remove residual serum or media components and again pelleted by low speed centrifugation. The cell pellets will then be disrupted or lysed using either chemical methods (such as detergents or enzymes, for example) or physical methods such as sonication, French press or high shear forces, such as rapid pipetting through small bore orifices like pipette tips. These lysed cell preparations may be fractionated by various centrifugation or fractionation procedures known in the art to obtain for example, fractions enriched for cellular membranes, intracellular organelles such as nuclei or mitochondria, higher-ordered oligomeric complexes, cytosolic components, or the like. The skilled artisan will recognize that such fractions may be further purified or fractionated using, for example, additional centrifugation, chromatographic, electrophoretic, or other fractionation techniques.

Once parallel fractions of the desired purity are obtained, these samples will be analyzed using conventional analytical techniques, such as electrophoresis or chromatography, and profiles generated. Comparison of wt cell profiles with deficient cell profiles will allow the identification of differentially expressed genes and/or modified proteins. For example, the radiolabeled phosphoprotein profiles visualized by autoradiography of 2-dimensional polyacrylamide gels will be different between parallel extracts from cultures of wt and GNK-deficient cells propagated in media containing γ-³²P-ATP, as sGNK will be radiolabeled in the wt cell extracts, but not the GNK-deficient cell extracts. Similarly, differential gene expression will be observed by the absence or enhancement of a particular polypeptide in one sample, but not the other.

### EXAMPLE 11

### Vascular Defects in GNK Deficient Mice

GNK deficient (GNK−/−) fetuses were obtained by crossing mice that were heterozygous for the GNK targeted mutation (GNK+/−), as described in Example 7. GNK deficiency resulted in lethality at approximately embryonic day 11.5 (e11.5). At a gross level, GNK deficient fetuses and yolk sacs appeared undervascularized. To examine the role of GNK in vascular development and function in more detail, the TIE2-lacZ transgene (Schlaeger T M et al (1997) Proc. Natl. Acad. Sci. 94:3058-3063) was moved onto the GNK deficient background. The lacZ transgene is expressed only in endothelial cells and thus provides a means of easily visualizing blood vessel structures during development.

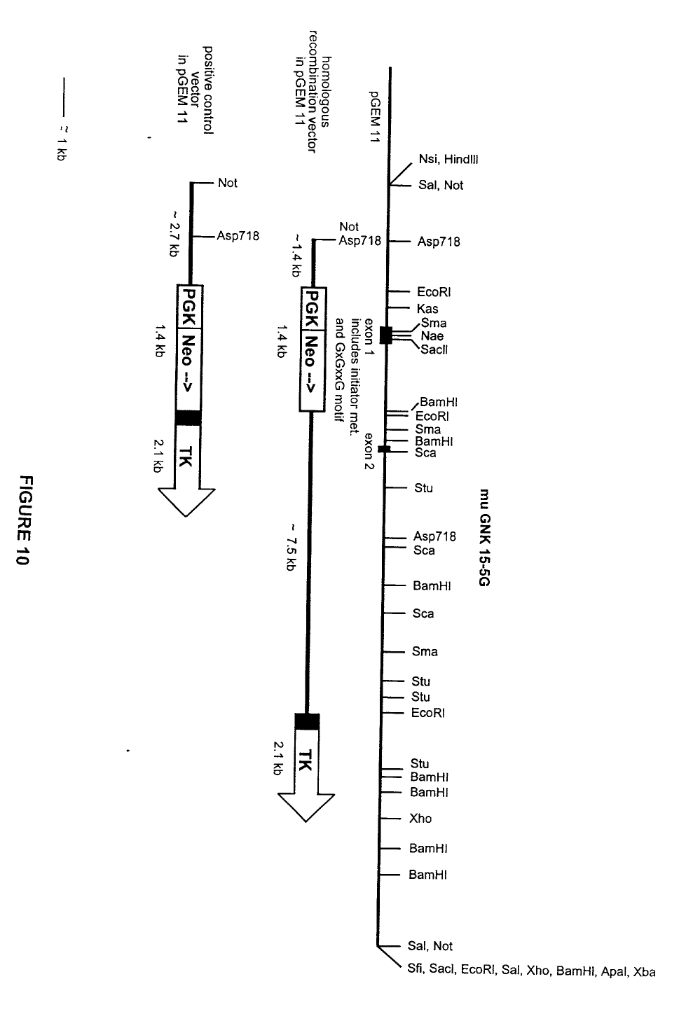

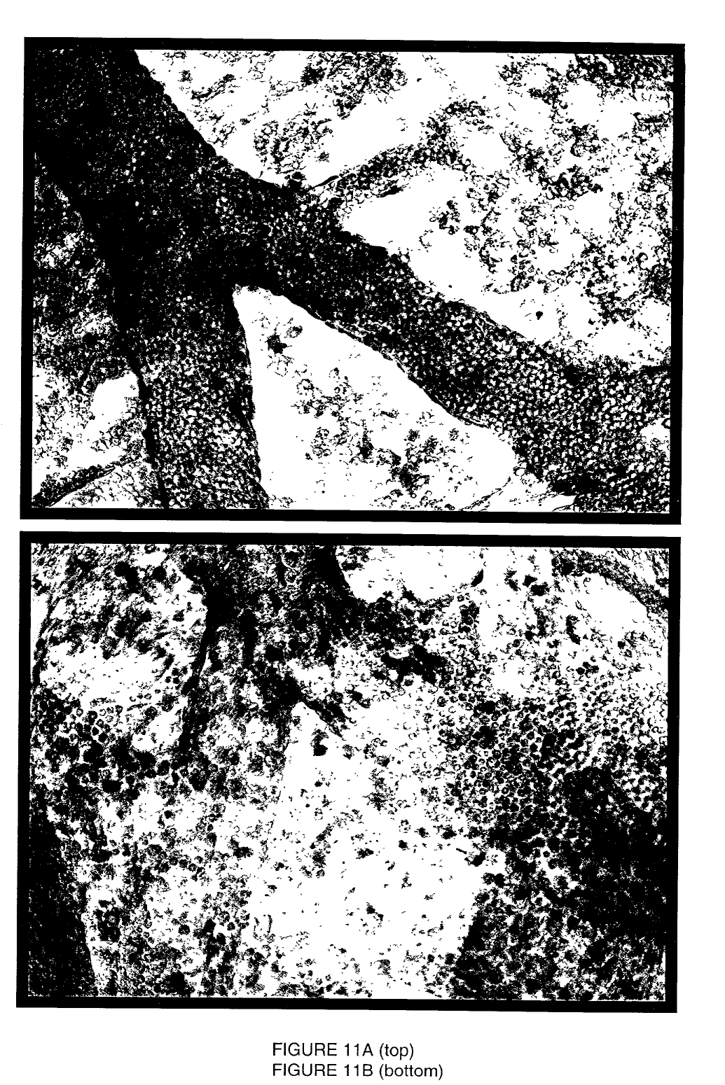

Vascular structures in TIE2-lacZ transgenic tissues that were either GNK deficient (GNK−/−) or GNK sufficient were visualized histochemically by staining for β-galactosidase activity essentially as described in Hogan et al. (Manipulating the mouse embryo: A laboratory manual, CSH Press, 1994). As shown in FIG. 11, yolk sacs derived from GNK−/−TIE2-lacZ+fetuses at e10.5 (FIG. 11B) lacked the organized vascular structures readily evident in e10.5 GNK sufficient TIE2-lacZ+yolk sacs (FIG. 11A).

Similar methods are used to analyze vascular structures in the yolk sac and the embryo proper at e9.5 and e11.5. To study the role of GNK in the development of functional endothelial cells, chimeric analyses are performed using the rosa26 system (Zambrowicz B P et al (1997) Proc. Natl. Acad. Sci. 94: 3789-3794). Briefly, GNK−/−ES cells are injected into rosa26 blastocysts. At various embryonic stages in adults, organs derived from the resulting chimeras are stained for β-galactosidase activity to determine which lineages are dependent upon GNK for development. In situ hybridization for GNK further demonstrates the temporal and spatial expression of GNK during development.

It will be apparent to those skilled in the art that various modifications and variations can be made in the disclosed methods and compositions without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An isolated nucleic acid molecule encoding sGNK or a variant thereof.

2. An isolated nucleic acid molecule of claim 1, comprising a sequence selected from the group consisting of (a) the sequence of nucleotides in SEQ ID NO: 1, from nucleotide 1 to nucleotide 4610; (b) nucleic acid molecules capable of hybridization to a nucleic acid molecule of (a) under conditions of moderate stringency, and which encode sGNK; and (c) nucleic acid molecules which are degenerate, as a result of the genetic code, with respect to the nucleic acid molecules of (a) or (b).

3. An isolated nucleic acid molecule of claim 1, comprising a sequence selected from the group consisting of (a) the sequence of nucleotides in SEQ ID NO: 1, from nucleotide 75 to nucleotide 2549; (b) nucleic acid molecules capable of hybridization to a nucleic acid molecule of (a) under conditions of moderate stringency, and which encode sGNK; and (c) nucleic acid molecules which are degenerate, as a result of the genetic code, with respect to the nucleic acid molecules of (a) or (b).

4. A recombinant expression vector comprising a promoter operably linked to a nucleic acid molecule according to claim 1, 2, or 3.

5. A host cell containing the recombinant expression vector of claim 4.

6. An isolated sGNK polypeptide.

7. An isolated sGNK polypeptide that is encoded by the nucleic acid of one of claims 1-3.

8. An isolated sGNK polypeptide comprising an amino acid sequence that is at least 80% identical to SEQ ID NO: 2.

9. The sGNK polypeptide of claim 8 comprising the sequence SEQ ID NO: 2.

10. An isolated GNK comprising a sufficient number of amino acids from SEQ ID NO: 4 to confer on the polypeptide vascularization regulatory activity on cells of mammalian origin.

11. An isolated nucleic acid molecule comprising a sufficient number of nucleotides from SEQ ID NO: 3 to encode a polypeptide that regulates vascularization.

12. A recombinant expression vector comprising a promoter operably linked to the nucleic acid sequence of claim 11.

13. A host cell containing the recombinant expression vector of claim 12.

14. A method for producing a polypeptide having vascularization regulating activity comprising culturing the recombinant host cell according to claim 13 under suitable conditions to express the polypeptide encoded by the nucleic acid molecule.

15. The method of claim 14 wherein the host cell is a bacterial, yeast, insect or mammalian cell.

16. The method of claim 14 wherein the host cell is a COS cell.

17. The method of claim 14 wherein the host cell is a 293 cell.

18. A method for stimulating blood vessel development comprising administering a therapeutically effective amount of GNK or GNK agonist to a patient.

19. The method of claim 18 wherein the stimulation of blood vessel development promotes wound healing.

20. The method of claim 18 wherein the stimulation of blood vessel development reduces cardiac dysfunction.

21. A method of reducing blood vessel development comprising administering a therapeutically effective amount of a GNK antogonist to a patient.

22. The method of claim 21 wherein the reduction of blood vessel development slows or prevents tumor development.

23. A method for treating a vascularization disorder comprising the step of administering to a patient a therapeutically effective amount of a GNK antagonist.

24. The method of claim 23 wherein the vascularization disorder is psoriasis.

25. The method of claim 23 wherein the vascularization disorder is arthritis.

26. The method of claim 23 wherein the vascularization disorder is proliferative retinopathy.

27. A pharmaceutical composition comprising GNK.

28. A pharmaceutical composition comprising at least one GNK agonist.

29. A pharmaceutical composition comprising at least one GNK antagonist.

30. An antibody molecule, or fragment thereof, which binds specifically to the vascularization regulatory domain of GNK.

31. An antibody molecule, or fragment thereof, which binds specifically to a portion of the sGNK of one of claims 6-9.

32. The antibody molecule, or fragment thereof, of claim 31 which is a polyclonal antibody.

33. The antibody molecule, or fragment thereof, of claim 31 which is a monoclonal antibody.

34. A method for substantially purifying sGNK comprising:
coupling the antibody molecule, or fragment thereof, of any of claims 30 to 33, to a solid support to form a matrix that selectively binds sGNK;
applying a mixture of polypeptides containing the sGNK to the matrix;
introducing conditions under which the sGNK and the antibody molecule, or fragment thereof, form an antigen-antibody complex;
washing unbound components of the mixture of polypeptides from the matrix; and
eluting the sGNK from the matrix.

35. A homologous recombination vector comprising a nucleotide sequence substantially similar to SEQ ID NO: 3, the sequence differing from SEQ ID NO: 3 by the addition, deletion, or substitution of one or more nucleotides to prevent expression of a polypeptide with vascularization regulatory capability, structurally linked to one or more selectable marker genes.

36. The homologous recombination vector of claim 35 wherein at least one selectable marker gene confers resistance to G418.

37. The homologous recombination vector of claim 35 wherein at least one selectable marker gene confers sensitivity to ganciclovir.

38. A method for generating GNK-deficient cells comprising:
transfecting cells with a homologous recombination vector that is incapable of expressing biologically active GNK and that contains a selectable marker;
selecting for transfected cells using selective medium;
propagating the transfected cells in culture; and
monitoring the propagated cells for GNK expression.

39. A method for identifying genes or gene products involved in regulating vascularization comprising:
propagating GNK-deficient cells or organs in parallel with wild-type cells or organs;
preparing parallel samples from the GNK-deficient and wild-type cells for analysis;
comparing the parallel GNK-deficient and wild-type samples for differential gene expression or protein modification; and
identifying the differentially expressed gene or differentially modified protein.

40. A method for identifying genes or gene products involved in regulating vascularization comprising:
propagating sGNK-deficient cells or organs in parallel with wild-type cells or organs;
preparing parallel samples from the sGNK-deficient and wild-type cells for analysis;
comparing the parallel sGNK-deficient and wild-type samples for differential gene expression or protein modification; and
identifying the differentially expressed gene or differentially modified protein.

41. A method of identifying a compound that modulates a protein-protein interaction between GNK and sGNK, comprising:
a) contacting a candidate compound with GNK and sGNK under conditions permitting the interaction between GNK and sGNK, and
b) measuring the ability of the candidate compound to modulate the interaction between GNK and sGNK.

42. A method of identifying a compound that modulates phosphorylation of sGNK by GNK, comprising:
a) contacting a candidate compound with GNK and sGNK under conditions permitting the phosphorylation of sGNK by GNK, and
b) measuring the ability of the candidate compound to modulate the phosphorylation of sGNK by GNK.

43. A method of identifying a compound that modulates vascularization comprising:
a) contacting a candidate compound with GNK or sGNK, and
b) measuring the ability of the candidate compound to modulate a biological activity of the GNK or sGNK.

44. A compound identifiable by a method according to one of claims 41-43.

45. The compound of claim 44 wherein the compound is selected from the group consisting of activators of GNK, inhibitors of GNK, activators of sGNK, inhibitors of sGNK, activators of an interaction between GNK and sGNK, inhibitors of the interaction between GNK and sGNK, activators of phosphorylation of sGNK by GNK, and inhibitors of phosphorylation of sGNK by GNK.

46. A nonhuman transgenic embryo, fetus, or animal that is heterozygous for a GNK targeted mutation.

47. A nonhuman GNK-deficient embryo or fetus produced by crossing heterozygous animals according to claim 46.

48. A cell from the embryo, fetus, or animal of claim 46 or 47.

49. A GNK deficient cell.

50. An sGNK deficient cell.

51. A recombinant vector comprising a promoter operably linked to a nucleic acid molecule encoding sGNK according to claim 8.

52. A host cell containing the recombinant expression vector of claim 51.
